(19) United States
(12) Patent Application Publication
(10) Pub. No.: US 2026/0257018 A1
(43) Pub. Date: Sep. 3, 2026

(54) SYSTEMS AND METHODS FOR BLOW-FILL-SEAL (BFS) TEAR-ACTIVATION VALVES

(71) Applicant: Koska Family Limited, East Sussex (GB)

(72) Inventors: Marc Andrew Koska, Dartmouth (GB); Jeff Price, Winter Garden, FL (US); Max Kingsley Hannon, London (GB)

(21) Appl. No.: 19/654,446

(22) Filed: Apr. 21, 2026

Related U.S. Application Data
(63) Continuation of application No. PCT/US24/52752, filed on Oct. 24, 2024.
(60) Provisional application No. 63/592,885, filed on Oct. 24, 2023.

(51) Int. Cl. A61M 5/28; A61J 1/06; A61M 5/31

(52) U.S. Cl. CPC A61M 5/285; A61J 1/067; A61M 5/282; A61M 2005/312

(57) ABSTRACT

A pre-filled medical delivery assembly configured to allow delivery of a single dose of a therapeutic agent (e.g., vaccine, drug, medicament, etc.) from a Blow-Fill-Seal (BFS) vial to a patient. The delivery assembly including a tear-activation valve that provides for an internal, mechanical BFS vial opening, and defines an Auto-Disable (AD) feature that prevents refilling or re-use of the assembly.

### CLAIM OF PRIORITY

The present Application is a Continuation Application of PCT Application No. PCT/US2024/052752 titled SYSTEMS AND METHODS FOR BLOW-FILL-SEAL (BFS) TEAR-ACTIVATION VALVES and filed on October 24, 2024, which PCT Application claims the benefit of priority of U.S. Provisional Application No. 63/592885 titled SYSTEMS AND METHODS FOR BLOW-FILL-SEAL (BFS) TEAR-ACTIVATION VALVES and filed on October 24, 2023. The entirety of each of these Applications is incorporated by reference herein for all purposes.

### BACKGROUND

Staggering numbers of people become infected, severely ill and/or die from a variety of diseases each year, some of which are preventable (or the severity of which could have been mitigated) through injectable medicines, medicaments, vaccines, drug products or other fluid agents (collectively "injectable medicines" herein, injectable medicines referring to fluid agents delivered via use of a syringe or other needle-type medical delivery device). Although injectable medicines have led to a decline in the number of cases of several infectious diseases (or the severity of symptoms or instances of death resulting therefrom) and/or ability to better manage ongoing health conditions such as diabetes, and have also become useful for administration of other medicaments such as contraceptives, the need for additional innovation in injectable medicines remains because the demand for effective contraceptive programs and control of existing and emerging diseases continues to grow and existing solutions leave room for further improvements. Applicant has previously invented various methods and systems intended to address the growing demand for injectable medicines through single-dose, pre-filled Blow-Fill-Seal (BFS) injector devices that comprise one or more benefits, such as (i) being manufacturable in a cost effective manner, in large quantities and at short notice to meet unanticipated peaks in demand; (ii) incorporating simplicity of design that lends itself to administration by users who have little or no medical training (e.g., are suitable for self-injection); and/or (iii) including features that minimize risk of re-use or otherwise prioritize safety concerns. Applicant continues to innovate in this field by developing various new and beneficial systems, devices and components that will provide additional options and opportunities for drug manufacturers to select a single-dose, pre-filled delivery system that best fits their needs.

### SUMMARY

In some embodiments, a BFS connection, injection, and/or administration assembly may comprise a connection, injection, and/or administration element or system formed and configured to mate with a BFS vial or bottle such that the contents of the BFS bottle may be injected into (and/or otherwise administered to) a patient.

BFS connection, injection, and/or administration assemblies may comprise, for example, (i) a BFS port formed at a first assembly or connection end, the BFS port comprising (a) a valve seat configured to accept an integral tear- activation valve member of a BFS bottle inserted into the BFS port and/or (b) threads configured to accept one or more mounting flanges (or other protrusion(s)) of the BFS bottle; and/or (ii) a canula bore extending between the BFS port at the first assembly or connection end of the assembly and a second or administration end of the assembly, the canula bore configured to accept and/or mount a canula, needle, needle hub, and/or other administration member to be in fluid communication with the BFS port, wherein in the case that the BFS bottle is inserted into the BFS port and the mounting flange(s) of the BFS bottle is engaged with the threads, the tear- activation valve portion of the BFS bottle is advanced into the valve seat. According to some embodiments, additional advancement of the BFS bottle into the BFS port may cause the tear-activation valve portion of the BFS bottle to engage with one or more shear projections, ridges, or features disposed within the valve seat. In some embodiments, the shear projection(s) may resist movement (e.g., rotational movement) of the tear-activation valve portion of the BFS bottle within the valve seat and cause the tear-activation valve portion of the BFS bottle to shear or tear (at least partially) from the BFS bottle, thereby exposing fluid contents of the BFS bottle to the valve seat and/or BFS port. In some embodiments, the tear-activation valve may permit fluid to be expelled from the BFS bottle, through the administration member and, e.g., to/into a patient. According to some embodiments, the tear- activation valve may function as a disablement device. Subsequent to administration, for example, should a user attempt to recharge or reintroduce fluid into the BFS bottle, the force of the fluid entering the valve seat/BFS port (and/or suction generated by an activation of a compressible reservoir) may cause the tear-activation valve portion of the BFS bottle (e.g., that is now only hingedly-attached to or detached from the BFS bottle) to press against or into the hole in the BFS bottle, thereby preventing reintroduction of fluid into the BFS bottle.

According to some embodiments, a BFS injection system may comprise a BFS vial or bottle formed with an integral tear-activation valve. One end of the BFS bottle may be molded with an integral projection forming the tear-activation valve and an attendant tear line or junction between the tear-activation valve and a seal of the BFS bottle, for example, in which shear force applied to the tear-activation valve may cause at least a portion of the tear- activation valve to separate from the BFS bottle, thereby opening the end of the BFS vial and providing access to the fluid stored therein. In some embodiments, the BFS bottle may be coupled to a medical delivery, injection, and/or coupling device and/or system that facilitates the separation (full or partial) of the tear-activation valve from the BFS bottle. According to some embodiments, the BFS bottle may comprise, (i) a compressible fluid reservoir; (ii) a neck portion in fluid communication with the compressible fluid reservoir and formed at a first end of the BFS bottle; (iii) an angled exterior flange formed on an exterior of the neck portion and being operable to rotationally engage with corresponding threads of a connection/injection assembly (e.g., a BFS port thereof); (iv) a fluid seal formed at the first end of the BFS bottle; (v) a tear-activation valve formed in communication with the fluid seal, wherein the tear-activation valve is operable to be sheared from the BFS bottle via application of rotational force, thereby providing access to the contents of the BFS bottle.

In some embodiments, a BFS injection system may comprise the BFS bottle and a BFS connection assembly that permits direct patient injection (e.g., of a single dose of medicament) via the BFS bottle. The BFS injection system may comprise, for example, a connector defining a first end and a second end, the first end defining a mounting socket comprising interior threads into which an angled exterior flange of the BFS bottle is axially and rotationally mated, whereby the mating causes a tear-activation valve of the BFS bottle to shear from a fluid seal of the BFS bottle, thereby exposing the contents of the BFS bottle to the connector, and the second end being coupled to a needle such that an application of radially inward force to a compressible fluid reservoir of the BFS bottle expels a dose of fluid stored in the compressible fluid reservoir through the needle. According to some embodiments, the angled exterior flange may comprise two angled exterior flanges disposed on opposite sides of the neck portion and each of the two angled exterior flanges extends between one eighth and less than one quarter along an outer circumference of the neck portion.

### BRIEF DESCRIPTION OF THE DRAWINGS

An understanding of embodiments described herein and many of the attendant advantages thereof may be readily obtained by reference to the following detailed description when considered with the accompanying drawings, wherein:

FIG. 1A, FIG. 1B, FIG. 1C, FIG. 1D, FIG. 1E, FIG. 1F, FIG. 1G, FIG. 1H, FIG. 11, FIG. 1J, and FIG. 1K are

various views of a pre-filled medical delivery assembly according to some embodiments;

FIG. 2A, FIG. 2B, FIG. 2C, FIG. 2D, FIG. 2E, FIG. 2F, FIG. 2G, FIG. 2H, FIG. 21, FIG. 2J, FIG. 2K, FIG. 2L

FIG. 2M, FIG. 2N, FIG. 20, FIG. 2P, and FIG. 2Q are various views of a pre-filled medical delivery assembly according to some embodiments;

FIG. 3A, FIG. 3B, FIG. 3C, FIG. 3D, FIG. 3E, FIG. 3F, FIG. 3G, and FIG. 3H are various views of a pre- filled BFS vial according to some embodiments;

FIG. 4A and FIG. 4B are perspective views of a pre-filled BFS vial according to some embodiments;

FIG. 5A, FIG. 5B, FIG. 5C, FIG. 5D, FIG. 5E, FIG. 5F, FIG. 5G, FIG. 5H, FIG. 51, and FIG. 5J are various views of a pre-filled BFS vial system according to some embodiments;

FIG. 6A, FIG. 6B, FIG. 6C, FIG. 6D, FIG. 6E, FIG. 6F, FIG. 6G, and FIG. 6H are various views of a BFS connector according to some embodiments;

FIG. 7A, FIG. 7B, and FIG. 7C are various views of a BFS connector according to some embodiments; and

FIG. 8A, FIG. 8B, FIG. 8C, FIG. 8D, and FIG. 8E are various views of a BFS connector according to some embodiments.

### DETAILED DESCRIPTION

### I. Introduction

Embodiments described herein provide systems and methods for pre-filled medical delivery devices and/or assemblies that overcome drawbacks of current delivery devices and methods. For example, the pre-filled medical delivery assemblies of some embodiments may include a Blow-Fill-Seal (BFS) vial or bottle coupled to a specialized collar or connector that facilitates coupling of an administration member (e.g., a needle) to the BFS vial. In some embodiments, such a pre-filled medical delivery assembly may be selectively actuated by engagement of the connector with a tear-activation valve of the BFS vial, which may cause the BFS vial to open and/or which may prevent the BFS vial from being refilled. Utilization of such systems that employ BFS vials with tear-activation valves may be advantageous and may address various shortcomings of previous systems. Utilization of a tear-activation valve as described herein, for example, may (i) reduce the likelihood of leakage, e.g., as may be experienced in the case that a BFS vial is punctured utilizing an external object, (ii) reduce the likelihood of BFS artifacts/particles becoming introduced into the fluid to be administered, and/or (iii) provide a multi-purpose activation and automatic deactivation mechanism. In some embodiments, a specially-configured BFS vial may provide some or all of these advantages in a manner that is not achievable via previous designs.

. Typical tear-to-open BFS vials, for example, provide large grip features that enable a user to easily twist- off or open a seal of the BFS vial, but are necessarily activated via two-handed operation in an open-air environment that may be subject to contamination. Quite differently, in some embodiments, a small tear-activation valve that is not easily directly manipulated by a human operator may be provided, and the tear-activation valve may be cooperatively sized with a seat and/or features of a mated connector such that selective engagement of the connector with the BFS vial may cause the tear-activation valve to open the BFS vial within a sterile cavity of the connector/delivery assembly. The tear-activation valve, unlike the typical tear-and-toss grip features of previous BFS vials, may in some embodiments not be discarded, but may instead remain housed in the cavity (e.g., between the BFS vial and an administration component of the connector/assembly) to function as a refill prevention valve, as described herein. In some embodiments, the tear-activation valve may comprise and/or be defined by a BFS neck geometry that is specially configured to both define a tear seam and define a tear feature geometry that permits a tear element to be re-engaged with the neck of the BFS vial, e.g., after separation.

### II. Pre-filled Medical Delivery Assemblies with Tear-Activation Valves

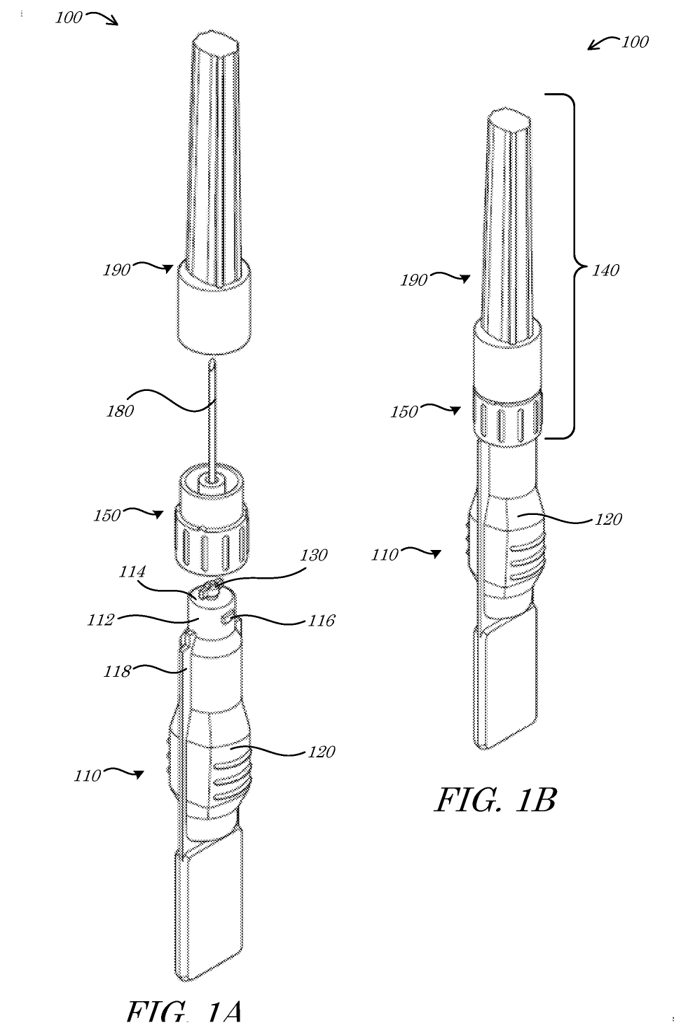

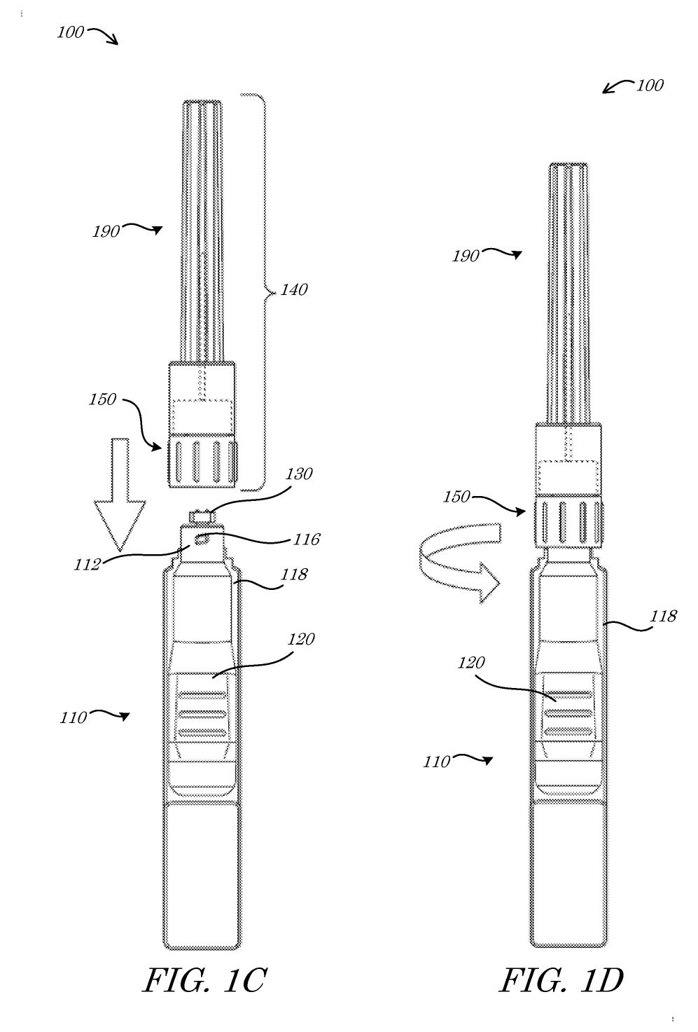

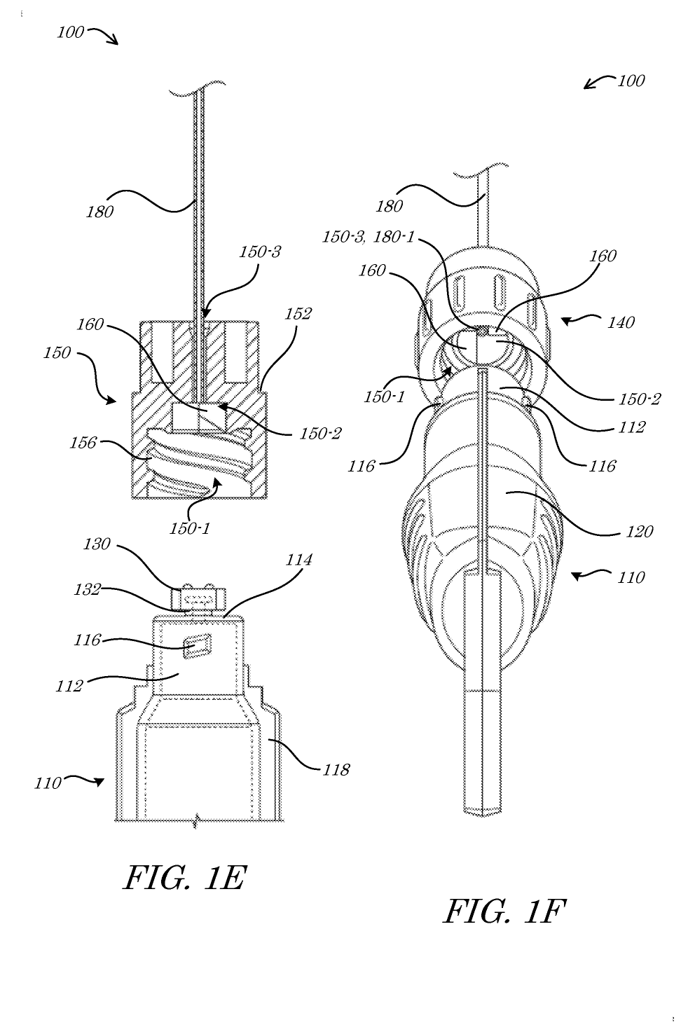

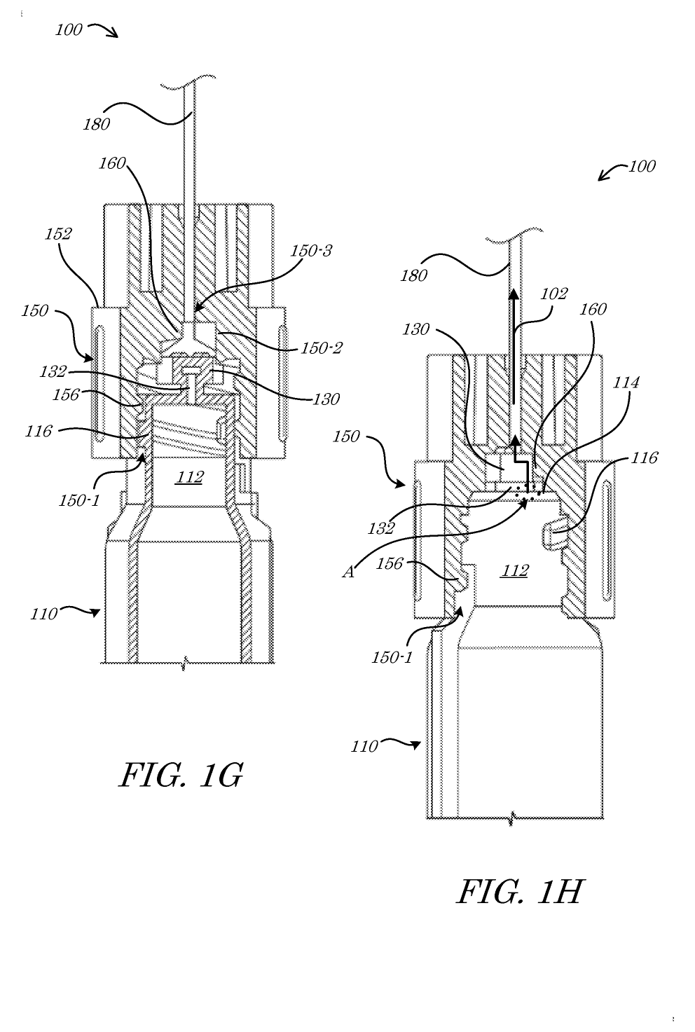

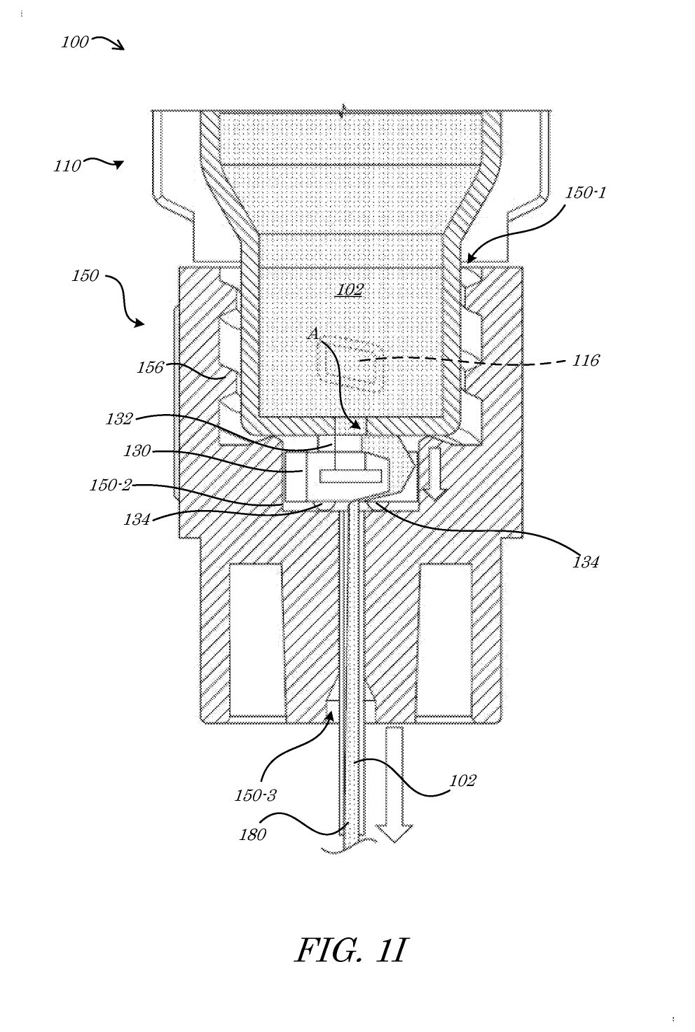

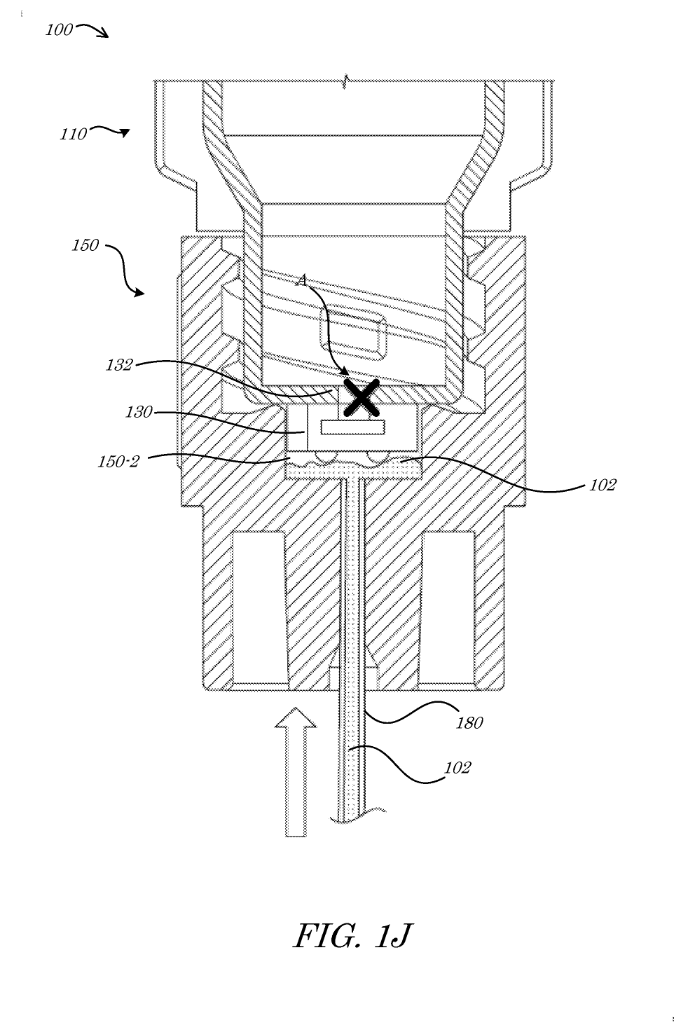

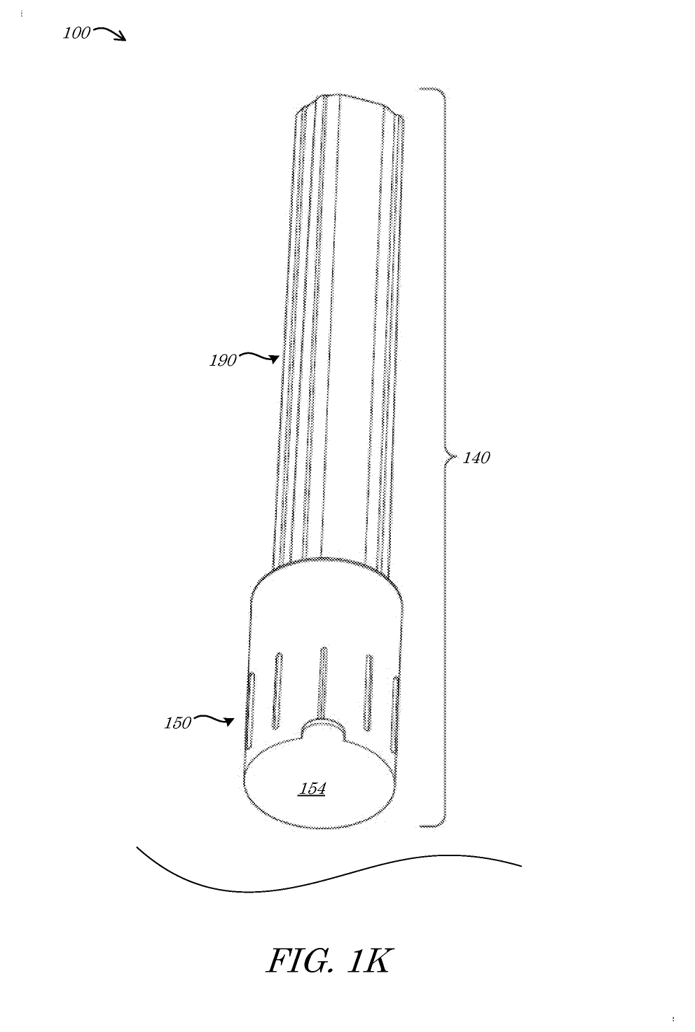

Referring initially to FIG. 1A, FIG. 1B, FIG. 1C, FIG. 1D, FIG. 1E, FIG. 1F, FIG. 1G, FIG. 1H, FIG. 11, FIG. 1J, and FIG. 1K, various views of a pre-filled medical delivery assembly 100 according to some embodiments are shown. In some embodiments, the pre-filled medical delivery assembly 100 may comprise various inter-connected and/or modular components configured to deliver a dose of fluid 102 (FIG. 1 H, FIG. 11, and FIG. 1J) to a patient (not shown), such as a BFS component, bottle, and/or vial 110 comprising and/or defining a vial neck 112, a fluid seal 114, a mounting flange 116, a side flange 118, and/or a collapsible and/or compressible reservoir 120. According to some embodiments, the BFS vial 110 may comprise an integral tear-activation valve 130, e.g., connected to the main body (not separately labeled) of the BFS vial 110 via a post 132, and/or comprising one or more standoffs 134 on a distal end thereof. In some embodiments, the pre-filled medical delivery assembly 100 may comprise an administration module, component, or assembly 140 comprising a connector 150. The connector 150 may comprise and/or define, for example, an interior volume, socket, or BFS port 150-1 disposed at a first or proximal end thereof. According to some embodiments, the BFS port 150-1 may comprise and/or define (e.g., on or in an interior surface thereof) a shaped valve seat 150-2 that is configured to accept the tear-activation valve 130 of the BFS vial 110 (e.g., in the case that the neck 112 of the BFS vial 110 is inserted into the BFS port 150- 1). In some embodiments, the connector 150 may comprise and/or define a canula bore 150-3 extending from a distal extent of the BFS port 150-1 (and/or the valve seat 150-2) to a distal or second end of the connector 150.

In some embodiments, the connector 150 may comprise and/or define an exterior (e.g., radial) flange 152 and/or may be maintained as a closed and/or sterile component via a seal 154 (e.g., a foil, wax, paper, and/or other thin, pierceable, tear-able, and/or removable object or layer coupled to the connector 150; FIG. 1K) That seals the BFS port 150-1. According to some embodiments, the connector 150 may comprise and/or define threads 156 (FIG. 1E, FIG. 1F, FIG. 1G, FIG. 1H, FIG. 11, and FIG. 1J) Formed and/or disposed within the BFS port 150-1. In some embodiments, the connector 150 (and/or the valve seat 150-2) may comprise and/or define one or more shear projections 160 (FIG. 1E, FIG. 1F, FIG. 1G, and FIG. 1H). According to some embodiments, the connector 150 may comprise and or house an administration member such as a canula or needle 180 (e.g., having a proximal end 180-1 disposed in or adjacent to, or otherwise in fluid communication with, the valve seat 150-2 and/or the BFS port 150-1) that is selectively shrouded by a cap 190. In some embodiments, the needle 180 may be disposed and/or housed in the canula bore 150-3 and/or the cap 190 may engage with and/or be selectively retained by the exterior flange 152 of the connector 150. In some embodiments, the administration assembly 140 (FIG. 1B, FIG. 1C, FIG. 1K) May comprise a pre-coupled grouping, assembly, and/or packaging of the connector 150, the needle 180 (and/or other administration member), the cap 190, and/or the seal 154.

According to some embodiments, and with reference to FIG. 1A, FIG. 1B, FIG. 1C, and FIG. 1D, the compressible reservoir 120 of the BFS vial 110 may be filled (fully or partially) with the fluid 102 or another agent (not separately shown in FIG. 1A, FIG. 1B, FIG. 1C, and FIG. 1D) to be delivered, e.g., to a patient (not shown). According to some embodiments, the fluid 102 may be injected into the BFS vial 110 in a sterile environment during manufacture via a BFS process and sealed within the BFS vial 110 via the fluid seal 114 (and/or by the formation of the tear-activation valve 130 and/or the post 132). The fluid seal 114 (and/or the tear-activation valve 130 and/or the post 132) may comprise a portion of the molded BFS vial 110 for example that is configured to be ruptured to expel the fluid 102, e.g., such as by (i) defining an area of reduced wall thickness (not shown; e.g., a rupture or failure feature, line, and/or point), (ii) providing a flat or planar surface operable to be compromised by a shearing of the tear-activation valve 130, and/or (iii) by being oriented normal to an axis of the BFS vial 110 (and/or the pre- filled medical delivery assembly 100). In some embodiments, the fluid seal 114 may comprise a foil, wax, paper, and/or other thin, object or layer coupled to the BFS vial 110. According to some embodiments, the neck 112 of the BFS vial 110 may comprise the mounting flange 116 such as, e.g., the angled exterior flanges depicted. The mounting flanges 116 may, for example, be shaped and/or sized to be operable to provide a selective engagement or fit with the threads 156 within the BFS port 150-1 of the connector 150.

In some embodiments, the pre-filled medical delivery assembly 100 may include a modular design consisting of separately constructed components 110, 140 cooperatively arranged and coupled to one another. As depicted in FIG. 1B, FIG. 1C, and FIG. 1K, for example, the BFS vial 110 and the administration assembly 140 may be manufactured, packaged, shipped, stored, and/or provided as separate components. In such a manner, the administration assembly 140 may not need to be stored or shipped in accordance with often restrictive requirements imposed on medicaments and may accordingly reduce the amount of space required for such specialized storage and/or shipping. The administration assembly 140 may also or alternatively be manufactured, stored, and/or shipped in advance (e.g., at a first time) while the BFS vial 110 that is pre-filled with the fluid 102 may be manufactured, stored, and/or shipped at a later time (e.g., a second time). In some embodiments, the delay between the first time and the second time may be lengthy without causing determinantal effects, as the administration assembly 140 may be stored, in some embodiments, indefinitely. In such a manner, units of the administration assembly 140 may be provided to be on hand in advance of the availability and/or arrival of the BFS vial 110, reducing supply chain constraints in the case of proactive administration assembly 140 procurement.

In some embodiments, the BFS vial 110 storing the fluid 102 (e.g., liquid agent) can be maintained as separate from other components of such a system or device (e.g., from the delivery or administration assembly 140) until ready for use, activation or administration to a patient. Maintaining components 110, 140 as separate may comprise, for example, (i) storing each in a distinct secondary packaging solution (not shown; e.g., a pre-filled BFS vial 110 may be stored in a foil pouch while a delivery assembly 140 to which the BFS vial 110 may be connected prior to administration of the fluid 102 (e.g., fluid agent) stored within it may be stored in cardboard cartons), or (ii) pre-assembling the components 110, 140 into a pre-activation mode but packaging them in the same secondary packaging solution (e.g., both a pre-filled BFS vial 110 and a corresponding administration assembly 140 may, in some embodiments, be packaged into the same foil pouch). In such embodiments, the seal 154 may not be included or may alternatively comprise the foil pouch or other seal/safety mechanism that encapsulates, retains, and/or houses the partially engaged BFS vial 110 and delivery assembly 140. According to some embodiments, the "pre-activation mode" or "transport" may comprise a partial or first degree of engagement of the mounting flange(s) 116 with the threads 156. The first degree of engagement may, for example, comprise a first amount of rotational and/or axial advancement that establishes a physical coupling of the BFS vial 110 and the administration assembly 140 (and/or the connector 150 thereof) but that does not reach a level of rotational and/or axial advancement sufficient to engage the tear-activation valve 130 with the shear projection 160. In some embodiments, additional engagement (e.g., a second degree of engagement) beyond the first degree of engagement may cause the shear projection 160 to engage with the tear-activation valve 130, thereby rupturing the fluid seal 114 and "activating" the pre-filled medical delivery assembly 100.

In some embodiments, the components 110,140 may be coupled, e.g., in the field and/or in situ, to provide an active pre-filled (e.g., injectable) medical delivery device. The seal 154 of the administration assembly 140 (as shown in FIG. 1K) may, for example, be removed from the administration assembly 140 and the administration assembly 140 (and/or the BFS port 150-1 of the connector 150 thereof) may be aligned with the neck 112 of the BFS vial 110, as shown in FIG. 1C. According to some embodiments, the administration assembly 140 may be axially engaged to couple with the BFS vial 110 via application of a mating axial force, as shown in FIG. 1C. The administration assembly 140 (and/or the connector 150 thereof) may be threaded onto the neck 112 of the BFS vial 110, as depicted in FIG. 1D for example, such that the cooperatively shaped threads 156 accept and/or guide the mounting flange(s) 116, thereby selectively and/or removably coupling the BFSvial 110 and the administration assembly 140.

As depicted in FIG. 1B, FIG. 1D, and FIG. 1G, the neck 112 of the BFS vial 110 may be threaded and/or otherwise urged into the BFS port 150-1 of the connector 150 until the tear-activation valve 130 becomes seated in (and/or coupled to or mated with) the valve seat 150-2 (e.g., a seated position). In such a manner, the fluid seal 114 may be advantageously positioned adjacent to the proximal end 180-1 of the needle 180 and/or the proximal end of the canula bore 150-3. In some embodiments, and as depicted in FIG. 1H and FIG. 11, continued advancement (e.g., rotational advancement) of the tear-activation valve 130 into the valve seat 150-2 may cause the tear-activation valve 130 to engage with one or more shear projections 160 extending into the valve seat 150- 2. In such a manner, the tear-activation valve 130 may be subjected to rotational shear (and/or other) forces that cause the tear-activation valve 130 and/or the post 132 to rip, tear, and/or shear from the BFS vial 110 and/or the fluid seal 114 thereof, e.g., at "A". Such compromising of the structural integrity of the tear-activation valve 130 (and/or the post 132) may cause, as depicted in FIG. 1H and FIG. 11, a fluid pathway to form between the compressible reservoir 120 (at "A") and the needle 180. According to some embodiments, and as depicted in FIG. 1 H and FIG. 11, activation of (e.g., application of radially inward or "squeeze" force to) the compressible reservoir 120 may then cause the fluid 102 to travel from the compressible reservoir 120, through the opening at "A", into the valve seat 150-2, and through the needle 180 (and/or other administration member). In some embodiments, pressure exerted by the fluid exiting the BFS vial 110 may force the fully or partially separated tear-activation valve 130 (and/or post 132) away from the opening "A", thereby permitting the fluid 102 to be expelled. In some embodiments, the standoffs 134 may prevent the tear-activation valve 130 from sealing-off or blocking the proximal end 180-1 of the needle 180 and/or the proximal end of the canula bore 150-3, e.g., maintaining a fluid pathway from the BFS vial 110 through the needle 180.

According to some embodiments, and as depicted in FIG. 1J, application of a reverse axial force or pressure, e.g., imparted by an attempt to reinflate or reform the compressible reservoir 120, may be applied to reintroduce the fluid 102 (the same fluid 102 that was expelled and/or a different fluid) into the needle 180. Such reverse force/pressure may permit some fluid 102 to re-enter the valve seat 150-2 (as depicted), but may also urge the tear-activation valve 130 (and/or the post 132) toward the BFS vial 110 to block or re-seal the opening "A". As depicted in FIG. 1J, for example, and in the case that the fluid seal 114 was originally compromised at the juncture of the fluid seal 114 and the post 132 (e.g., at "A"), pressure (toward the BFS vial 110 as applied by the fluid 102 and/or suction force applied by the BFS vial 110) may cause the post 132 to enter the hole/opening "A", thereby preventing the fluid 102 from entering/re-entering the BFS vial 110. In such a manner, for example, the existence of the tear-activation valve 130 between the BFS vial 110 and the needle 180 (e.g., as disposed or "trapped" in the valve seat 1250-) may comprise an Auto-Disable (AD) device that becomes active at the same time that the pre- filled medical delivery assembly 100 is activated for use (e.g., by fully-engaging/coupling the BFS vial 110 with the administration assembly 140 and thereby causing the tear-activation valve 130 (and/or the post 132) to form the opening "A"). In some embodiments, an additional one-way valve (not shown; e.g., a float and/or "bullet" valve) may be disposed in the fluid path (e.g., within a valve chamber formed in or by the connector 150; not shown) between the BFS vial 110 and the needle 180 to further prevent re-filling or re-use of the pre-filled medical delivery assembly 100 (and/or components thereof such as the BFS vial 110 and/or the administration assembly 140).

In some embodiments, the needle 180 may comprise a needle shaped and/or sized for at least one of subcutaneous, intramuscular, intradermal, and intravenous injection of the fluid agent into the patient. For ease of explanation and description, the figures and the description herein generally refer to the needle 180 as a needle. However, it should be noted that, in other embodiments, the needle 180 may also or alternatively comprise a nozzle (not shown) configured to control administration of the fluid agent to the patient. The nozzle may comprise a spray nozzle, for example, configured to facilitate dispersion of the fluid agent into a spray. Accordingly, a version of the administration assembly 140 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a version of the administration assembly 140 fitted with a droplet nozzle may be useful in the administration of a fluid agent by way of droplets, such as administration to the eyes, topical administration, and the like.

The fluid 102 or drug agent may include any type of agent to be injected into a patient (e.g., mammal, either human or non-human) and capable of producing an effect (alone, or in combination with an active ingredient). Accordingly, the agent may include, but is not limited to, a vaccine, a drug, a therapeutic agent, a medicament, a diluent, an active ingredient, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components such as the BFS vial 110 and/or the administration assembly 140.

According to some embodiments, the administration assembly 140 may be composed of a medical grade material. In some embodiments, the administration assembly 140 may be composed of a thermoplastic polymer or other "hard" plastic (e.g., greater than eighty (80) on the Rockwell "R" scale), including, but not limited to, polybenzimidazole, acrylonitrile butadiene styrene (ABS), polystyrene, polyvinyl chloride, or the like. In some embodiments, the pre-filled medical delivery assembly 100 may be advantageously manufactured (in mass quantities) in separate parts or portions, namely, at least the "soft" plastic BFS vial 110 portion (e.g., a "first" piece) and the "hard" plastic administration assembly 140 (e.g., the "second" piece), with such different plastic parts/portions being selectively coupled to administer a medication to a patient.

In some embodiments, fewer or more components 102, 110, 112, 114, 116, 118, 120, 130, 132, 134, 140, 150, 150-1,150-1, 150-2, 150-3, 152, 154, 156, 160, 180, 180-1, 190 and/or various configurations of the depicted components 102, 110, 112, 114, 116, 118, 120, 130, 132, 134, 140, 150, 150-1,150-1, 150-2, 150-3, 152,154, 156, 160, 180, 180-1, 190 may be included in the pre-filled medical delivery assembly 100 without deviating from the scope of embodiments described herein. In some embodiments, the components 102, 110, 112, 114, 116,118, 120,130, 132,134, 140,150, 150-1,150-2,150-3, 152,154, 156,160, 180, 180-1,190 may be similar in configuration and/or functionality to similarly named and/or numbered components as described herein. According to some embodiments, the pre-filled medical delivery assembly 100 may comprise only the BFS vial 110, only the administration assembly 140, or only the connector 150.

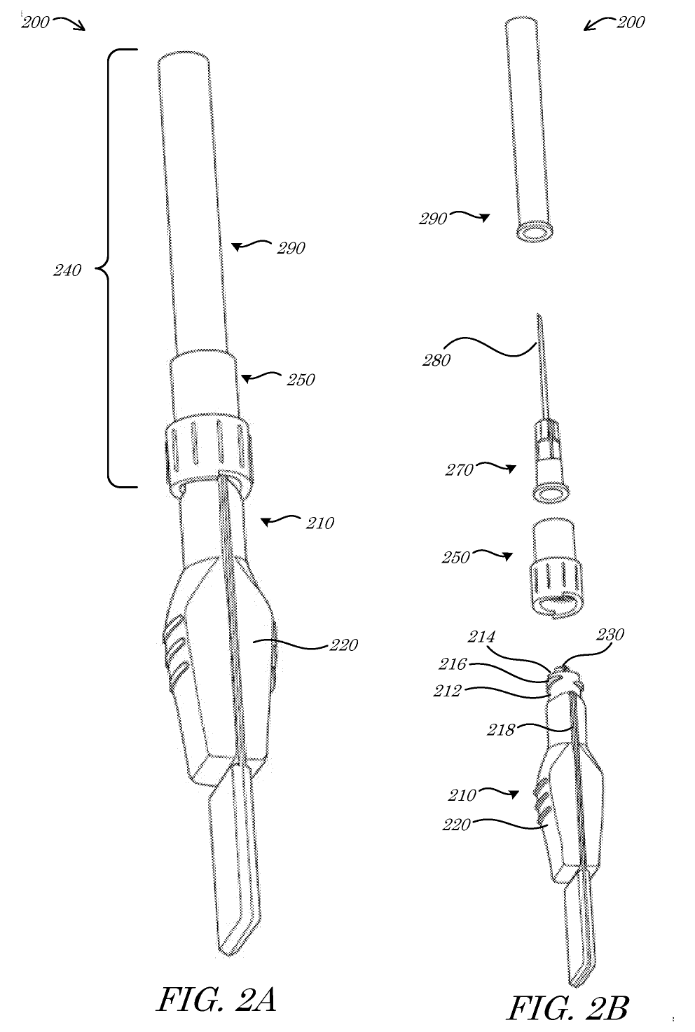

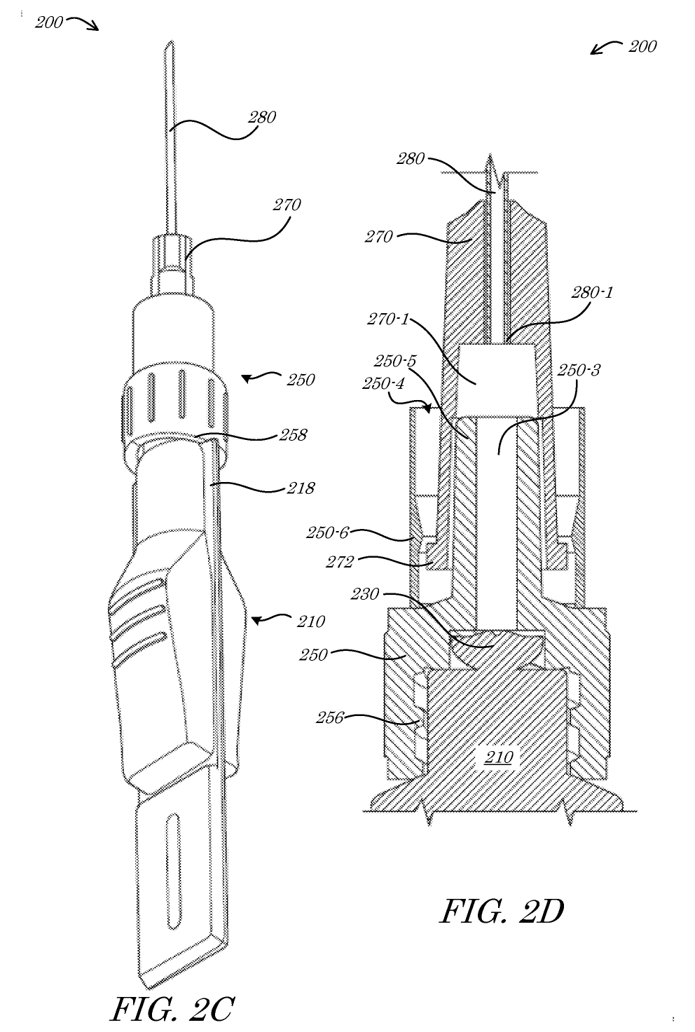

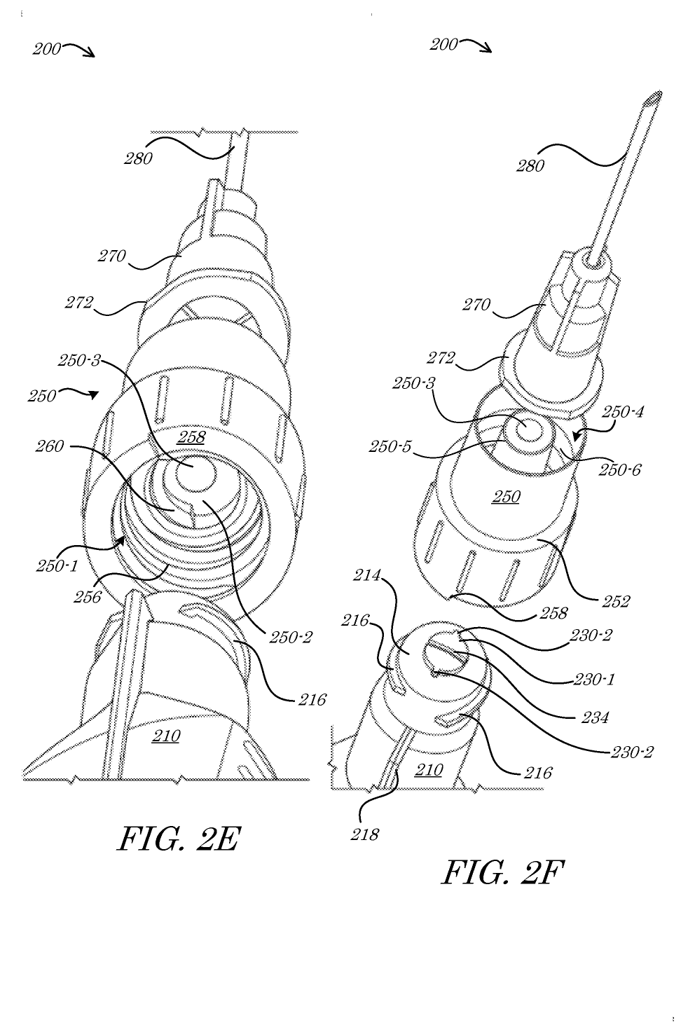

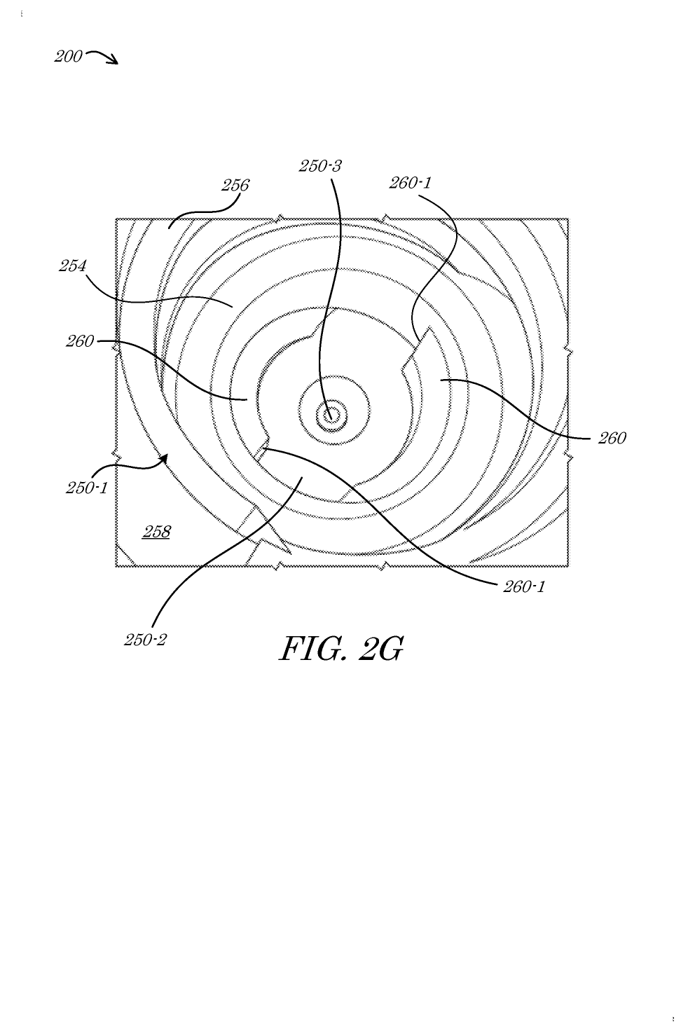

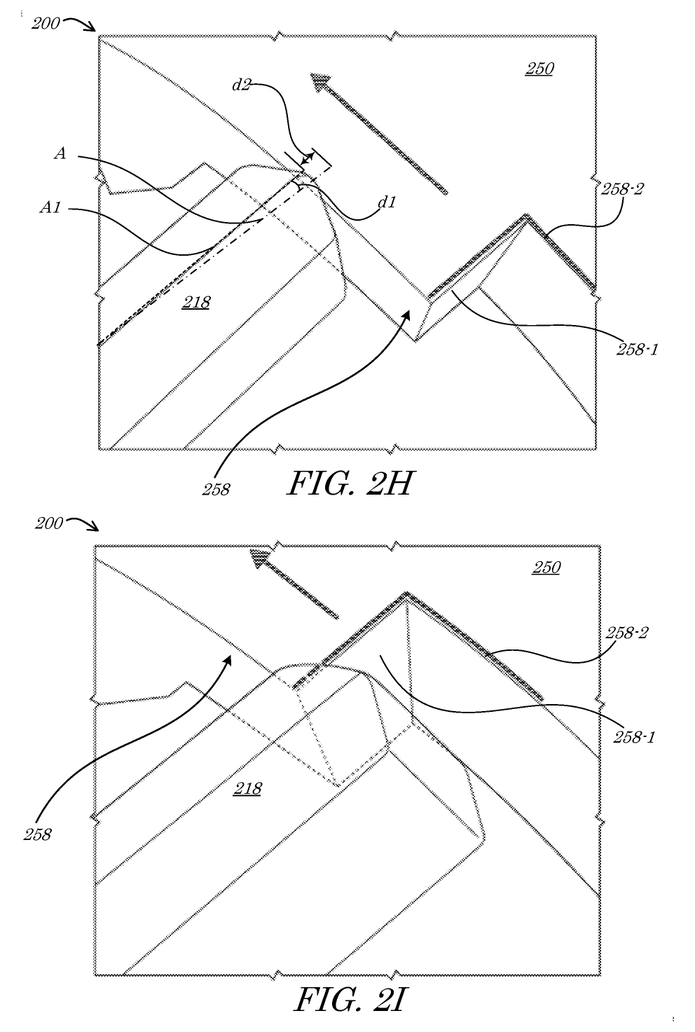

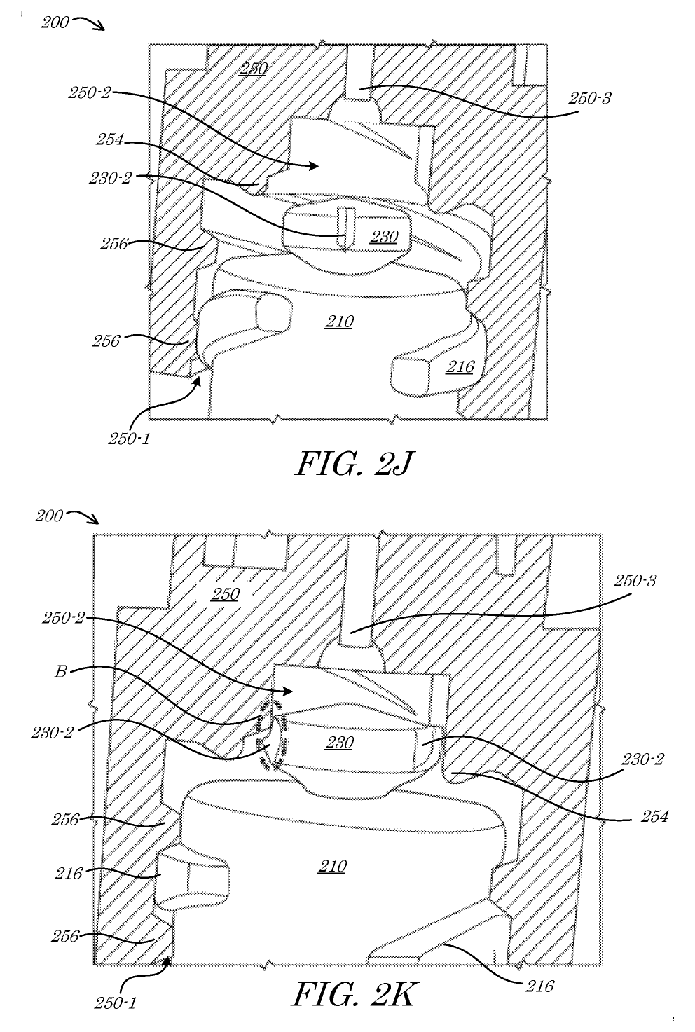

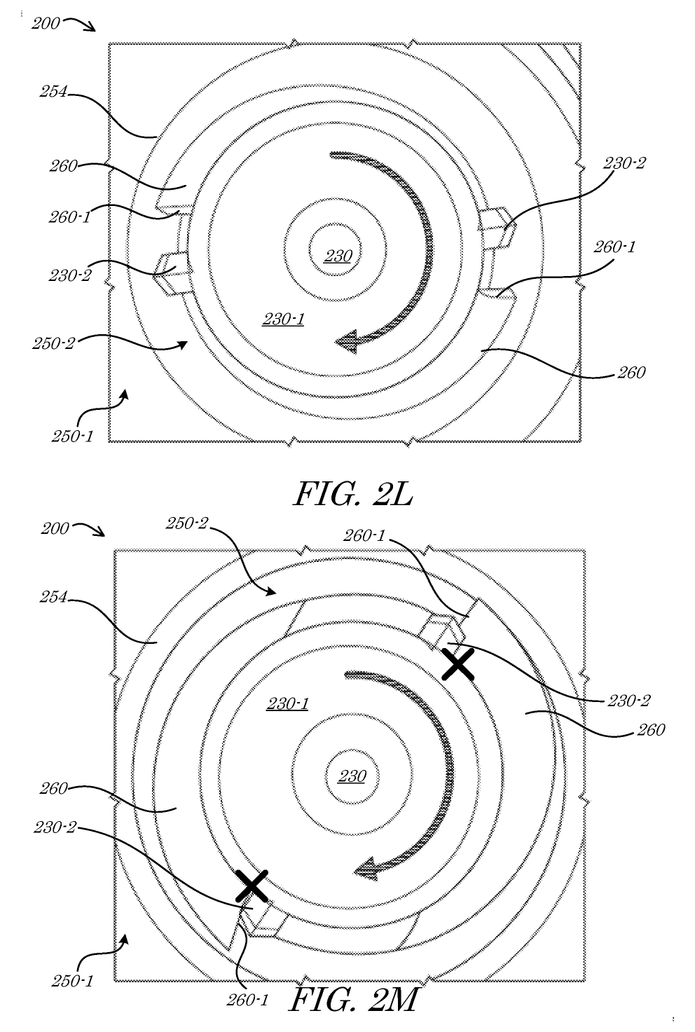

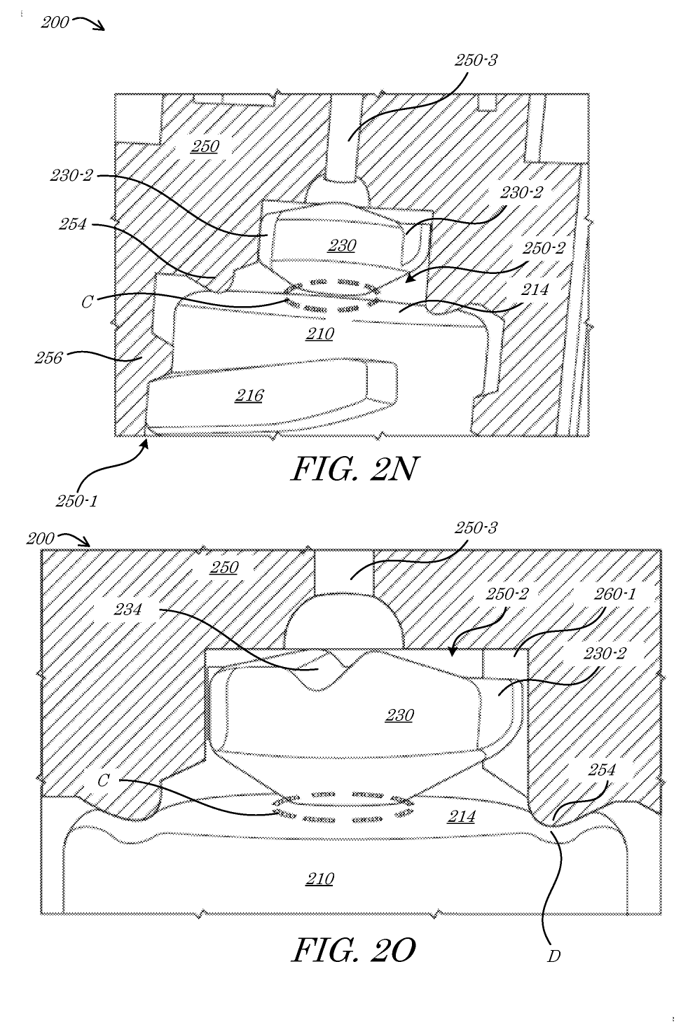

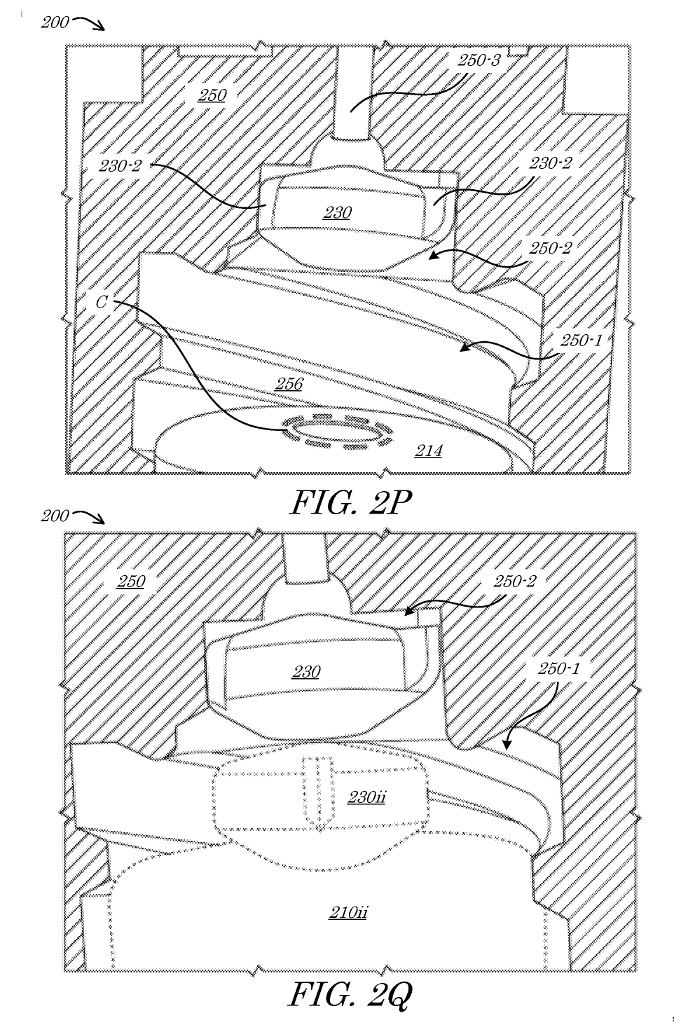

Turning to FIG. 2A, FIG. 2B, FIG. 2C, FIG. 2D, FIG. 2E, FIG. 2F, FIG. 2G, FIG. 2H, FIG. 21, FIG. 2J, FIG. 2K, FIG. 2L, FIG. 2M, FIG. 2N, FIG. 0, FIG. 2P, and FIG. 2Q, various views of a pre-filled medical delivery assembly 200 according to some embodiments are shown. In some embodiments, the pre-filled medical delivery assembly 200 may comprise various inter-connected and/or modular components configured to deliver a dose of fluid (not shown; e.g., the fluid 102 of FIG. 1H, FIG. 11, and FIG. 1J herein) to a patient (also not shown), such as a BFS component, bottle, and/or vial 210 comprising and/or defining a vial neck 212, a fluid seal 214, a mounting flange 216, a side flange 218, and/or a collapsible or compressible reservoir 220. According to some embodiments, the BFS vial 210 may comprise an integral tear-activation valve 230, e.g., connected to and/or formed on the main body (not separately labeled) of the BFS vial 210. In some embodiments, the tear-activation valve 230 may comprise and/or define a first shaped portion such as a circular nub 230-1 (e.g., as shown in FIG. 2F, FIG. 2L, and FIG. 2M) and/or one or more second shaped portions or "ears" 230-2 emanating radially therefrom. According to some embodiments, the tear-activation valve 230 may comprise and/or define a channel 234 formed and/or disposed on a distal end thereof (e.g., as depicted in FIG. 2F and FIG. 20). In some embodiments, the pre-filled medical delivery assembly 200 may comprise an administration module, component, or assembly 240 comprising a connector 250. The connector 250 may comprise and/or define, for example, an interior volume, socket, or BFS port 250-1 disposed at a first or proximal end thereof. According to some embodiments, the BFS port 250-1 may comprise and/or define (e.g., on or in an interior surface or area thereof) a shaped valve seat 250-2 that is configured to accept the tear-activation valve 230 of the BFS vial 210 (e.g., in the case that the neck 212 of the BFS vial 210 is inserted into the BFS port 250-1). In some embodiments, the connector 250 may comprise and/or define a fluid bore 250-3 extending from a distal extent of the BFS port 250-1 (and/or the valve seat 250-2) to a distal or second end of the connector 250.

In some embodiments, the connector 250 may comprise and/or define an interior volume, seat, or hub socket 250-4 disposed at the second or distal end thereof. According to some embodiments, the connector 250 may comprise and/or define a mounting projection, feature, or cone 250-5 that extends axially within (or at least partially within) the hub socket 250-4. As depicted, e.g., in FIG. 2D and FIG. 2F, the fluid bore 250-3 may extend through the mounting cone 250-5. In some embodiments, the connector 250 may comprise and/or define an interior radial flange, ridge, or lip 250-6 within the hub socket 250-4. The lip 250-6 may, for example, reduce an interior diameter of the hub socket 250-4 at the position of the lip 250-6 within the hub socket 250-4. In some embodiments, the connector 250 may comprise and/or define an exterior (e.g., radial) flange 252 and/or may be maintained as a closed and/or sterile component via a seal (not shown; e.g., a foil, wax, paper, and/or other thin, pierceable, tear- able, and/or removable object or layer coupled to the connector 250) that seals the BFS port 250-1. According to some embodiments, the connector 250 may comprise and/or define one or more of (i) a seal flange 254 formed and/or disposed within the BFS port 250-1 (FIG. 2G, FIG. 2J, FIG. 2K, FIG. 2L, FIG. 2M, FIG. 2N, and FIG. 20) and/or (ii) threads 256 (FIG. 2E, FIG. 2G, FIG. 2J, FIG. 2K, and FIG. 2N) formed and/or disposed within the BFS port 250-1.

According to some embodiments, the connector 250 may comprise and/or define a shaped edge 258 (FIG. 2E, FIG. 2G, FIG. 2H, and FIG. 21) at the proximal end, e.g., at the opening of the BFS port 250-1. The shaped edge 258, instead of comprising a uniform circumferential surface disposed at a uniform distance from the distal end of the connector 250, for example, may comprise and/or define a track or cam surface that varies in axial distance from the distal end of the connector 250 (and/or from the exterior flange 252). In some embodiments, the shaped edge 258 may comprise and/or define an anti-rotation surface 258-1 that comprises a surface normal to the circumferential plane of the proximal end of the connector 250. The anti-rotation surface 258-1 may be formed and/or defined, for example at a point where the furthest axial distance from the shaped edge 258 to the distal end of the connector (and/or to the exterior flange 252) transitions to the nearest axial distance from the shaped edge 258 to the distal end of the connector (and/or to the exterior flange 252). This transition may define, for example, a notch 258-2, as best illustrated in FIG. 2H and FIG. 21), that may be utilized to selectively engage with and/or retain the side flange 218 of the BFS vial 210.

In some embodiments, the connector 250 (and/or the valve seat 250-2) may comprise and/or define one or more internal protrusions, features, or shear projections 260 (FIG. 2E, FIG. 2G, FIG. 2L, and FIG. 2M). The shear projections 260 may, for example, interrupt an internal diameter of the valve seat 250-2 and/or may form or define respective shear surfaces 260-1 (FIG. 2G, FIG. 2L, FIG. 2M, and FIG. 20), e.g., projecting normally from an end-plane of the valve seat 250-2. According to some embodiments, the connector 250 may couple to a needle hub 270 that comprises and/or defines a socket 270-1 and/or an exterior flange 272. In some embodiments, the needle hub 270 may comprise and or house an administration member such as a canula or needle 280 (e.g., having a proximal end 280-1 disposed in or adjacent to, or otherwise in fluid communication with, the socket 270-1). According to some embodiments, the needle 280 (and/or other administration member such as a nozzle or dropper) may be selectively shrouded by a cap 290. In some embodiments, the needle 280 may be formed, disposed, and/or housed such that a mating of the socket 270-1 of the needle hub 270 with the mounting cone 250-5 of the connector 250 causes the fluid bore 250-3 of the connector 250 to be aligned and/or in fluid communication with the needle 280. An insertion of the mounting cone 250-5 into the socket 270-1 may, in some embodiments, cause the exterior flange 272 to pass axially inward beyond the lip 250-6 within the hub socket 250-4, thereby retaining the exterior flange 272 (and the needle hub 270) within the hub socket 250-4. The sizing of the exterior flange 272 and the minimum diameter of the hub socket 250-4 as defined by the lip 250-6 may be configured, for example, such that an amount of axial force required to insert and/or remove the needle hub 270 from the connector 250 may exceed a predetermined threshold, e.g., thirty-four Newtons (34 N).

In some embodiments, the cap 290 may engage with and/or be selectively retained by the exterior flange 252 of the connector 250. In some embodiments, the administration assembly 240 (FIG. 2A) may comprise a pre- coupled grouping, assembly, and/or packaging of the connector 250, the needle hub 270, the needle 280 (and/or other administration member), and/or the cap 290. In some embodiments, the administration assembly 240 may be sealed and/or packaged (not shown) to maintain sterility thereof, e.g., until use.

..According to some embodiments, the compressible reservoir 220 of the BFS vial 210 may be filled (fully or partially) with the fluid or other agent (not separately shown) to be delivered, e.g., to the patient. According to some embodiments, the fluid may be injected into the BFS vial 210 in a sterile environment during manufacture via a BFS process and sealed within the BFS vial 210 via the fluid seal 214 (and/or by the formation of the tear-activation valve 230). The fluid seal 214 (and/or the tear-activation valve 230) may comprise a portion of the molded BFS vial 210 for example that is configured to be ruptured to expel the fluid, e.g., such as by (i) defining an area of reduced wall thickness (not shown; e.g., a rupture or failure feature, line, and/or point), (ii) providing a flat or planar surface operable to be compromised by a shearing of the tear-activation valve 230, and/or (iii) by being oriented normal to an axis of the BFS vial 210 (and/or the pre-filled medical delivery assembly 200). In some embodiments, the fluid seal 214 may comprise a foil, wax, paper, and/or other thin, object or layer coupled to the BFS vial 210. According to some embodiments, the neck 212 of the BFS vial 210 may comprise the mounting flange 216 such as, e.g., the angled exterior flanges or tracks as depicted. The mounting flanges 216 may, for example, be shaped and/or sized to be operable to provide a selective engagement or fit with the threads 256 within the BFS port 250-1 of the connector 250.

In some embodiments, the pre-filled medical delivery assembly 200 may include a modular design consisting of separately constructed components 210, 240 cooperatively arranged and coupled to one another. According to some embodiments, the BFS vial 210 and the administration assembly 240 may be manufactured, packaged, shipped, stored, and/or provided as separate components. In such a manner, the administration assembly 240 may not need to be stored or shipped in accordance with often restrictive requirements imposed on medicaments and may accordingly reduce the amount of space required for such specialized storage and/or shipping. The administration assembly 240 may also or alternatively be manufactured, stored, and/or shipped in advance (e.g., at a first time) while the BFS vial 210 that is pre-filled with the fluid may be manufactured, stored, and/or shipped at a later time (e.g., a second time). In some embodiments, the delay between the first time and the second time may be lengthy without causing determinantal effects, as the administration assembly 240 may be stored, in some embodiments, indefinitely. In such a manner, units of the administration assembly 240 may be provided to be on hand in advance of the availability and/or arrival of the BFS vial 210, reducing supply chain constraints in the case of proactive administration assembly 240 procurement.

In some embodiments, as depicted in FIG. 2A for example, the components 210, 240 may be coupled, e.g., upon packaging, in the field, and/or in situ, to provide a pre-filled (e.g., injectable) medical delivery device in a first or inactive state. The neck 212 of the BFS vial 210 may be pre-inserted into the BFS port 250-1 of the connector 250, for example, and the mounting flange(s) 216 may be partially engaged with the threads 256 of the connector 250. According to some embodiments, the administration assembly 240 may be axially engaged to couple with the BFS vial 210 via application of a mating rotational and/or axial force, e.g., during an assembly process. The administration assembly 240 (and/or the connector 250 thereof) may be threaded onto the neck 212 of the BFS vial 210, for example, such that the cooperatively shaped threads 256 accept and/or guide the mounting flange(s) 216, thereby selectively and/or removably coupling the BFS vial 210 and the administration assembly 240.

As depicted in FIG. 2D, the neck 212 of the BFS vial 210 may be threaded and/or otherwise urged into the BFS port 250-1 of the connector 250 until the tear-activation valve 230 becomes seated in (and/or coupled to or mated with) the valve seat 250-2 (e.g., a seated position). In such a manner, the fluid seal 214 may be advantageously positioned adjacent to the proximal end of the fluid bore 250-3 (and/or the proximal end 280-1 of the administration member 280). In some embodiments, and as depicted sequentially in FIG. 2J, FIG. 2K, FIG. 2L, FIG. 2M, FIG. 2N, and FIG. 20, advancement (e.g., rotational and/or axial advancement) of the BFS vial 210 into the BFS port 250-1 may be conducted by (i) assembly of the pre-filled medical delivery assembly 200 to the first or transport state (via a first degree of advancement) and then (ii) selective activation of the pre-filled medical delivery assembly 200 (via a second degree of advancement). As depicted in FIG. 2J, for example, the BFS vial 210 may be inserted into the BFS port 250-1 and threaded therein (e.g., with at least one mounting flange 216 engaging with at least a first or lower portion of the threads 256). As depicted in FIG. 2K, advancement of the threads/insertion (e.g., with at least one mounting flange 216 further engaging with at least a second or middle portion of the threads 256) may cause the tear-activation valve 230 to enter the valve seat 250-2. In some embodiments, as shown, radial extents of the ears 230-2 may be greater than the interior diameter or extents of the valve seat 250-2, such that movement of the tear-activation valve 230 into the valve seat 250-2 causes the ears 230-2 to deform and/or compress, at "B". In some embodiments, the connector 250 may be constructed of injection-molded plastic that is harder than the BFS-produced plastic product that defines the BFS vial 210 and the respective ears 230-2 thereof, such that the ears 230-2 readily deform when forced into the valve seat 250-2.

According to some embodiments, the first or transport state may comprise the BFS vial 210 being advanced to a first degree of threading into the connector 250 such that the tear-activation valve 230 is positioned within the valve seat 250-2, but such that the tear-activation valve 230 has not yet engaged with the shear projection(s) 260, and the BFS vial 210 accordingly remains intact (e.g., FIG. 2K and/or FIG. 2L). With reference to FIG. 2L, for example, a cross-sectional view showing the tear-activation valve 230 portion of the BFS vial 210 within the BFS port 250-1 depicts the ears 230-2 of the tear-activation valve 230 having not yet engaged with the shear projections 260. In some embodiments, continued advancement (e.g., rotational advancement) of the BFS vial 210 beyond this first state and/or threaded position (e.g., with at least one mounting flange 216 engaging with at least a third or upper portion of the threads 256) may cause the tear-activation valve 230 to engage with one or more shear projections 260 extending into the valve seat 250-2. As depicted in FIG. 2M, for example, in the case that the threading is advanced to a second state, the ears 230-2 of the tear-activation valve 230 engage with the shear surfaces 260-1, that inhibit the further rotation of the ears 230-2 (as depicted by the "X" notations in FIG. 2M).

In some embodiments, the threads 256 may be configured such that achievement of the second state coincides with (or occurs in concert with) a rotational engagement of the shaped edge 258 of the connector 250 with the side flange(s) 218 of the BFS vial 210. As depicted in FIG. 2H, for example, an axially lengthened portion of the shaped edge 258 may rotationally align with and engage or interfere with a side flange 218. According to some embodiments, the engagement may cause a deformation and/or misalignment of the side flange 218. The side flange 218 may be elastically bent, for example, to deviate from a primary axial axis "A" to a bent or diverted axis "A1", as shown in FIG. 2H. The softer plastic of the side flange 218 may be forced by the shaped edge 258 to undergo a radial or circumferential twisting by an angular amount "d1", for example, and/or an axial shortening or compression by an amount "d2". In some embodiments, as the rotational engagement of the BFS vial 210 and the connector 250 continues, and as depicted in FIG. 21, the side flange 218 may be freed by being exposed to an axially shortened portion of the shaped edge 258 where the anti-rotation surface 258-1 and the notch 258-2 are disposed. According to some embodiments, the side flange 218 may, upon being exposed to and/or aligned with the notch 258-2, elastically rebound to the original axis "A" and to an original axial length, such that the side flange 218 becomes housed and/or disposed in the notch 258-2. In some embodiments, once the side flange 218 is disposed in the notch 258-2, counterrotation (e.g., an attempt to remove the connector 250 from the BFS vial 210) may be prevented by engagement of the side flange 218 with the anti-rotation surface 258-1, which, e.g., may be normal to the path of counterrotation. According to some embodiments, reconstitution of the side flange 218 into the notch 258-2 may produce an audible and/or tactile report that is operable to be recognized by a user, such that they may be provided with feedback acknowledging that the BFS vial 210 has been successfully engaged to a third state and/or position that has activated the tear-activation valve 230.

With reference to FIG. 2N, for example, upon achieving the third state of threading or engagement, the tear-activation valve 230 may be subjected to rotational shear (and/or other) forces that cause the tear-activation valve 230 to rip, tear, and/or shear from the BFS vial 210 and/or the fluid seal 214 thereof, e.g., forming a tear or hole at "C" in FIG. 2N and FIG. 20. Such compromising of the structural integrity of the tear-activation valve 230 may cause, a fluid pathway to form between the compressible reservoir 220 (at "C") and the needle 280 (e.g., via the fluid bore 250-3). According to some embodiments, activation of (e.g., application of radially inward or "squeeze" force to) the compressible reservoir 220 may then cause the fluid to travel from the compressible reservoir 220, through the opening at "C", into the valve seat 250-2, through the channel 234, into the fluid bore 250-3, and through the needle 280 (and/or other administration member). In some embodiments, pressure exerted by the fluid exiting the BFS vial 210 may force the fully or partially separated tear-activation valve 230 away from the opening "C", thereby permitting the fluid to be expelled. In some embodiments, the channel 234 may prevent the tear-activation valve 230 from sealing-off or blocking the proximal end of the fluid bore 250-3, e.g., maintaining a fluid pathway from the BFS vial 210 through the needle 280.

In some embodiments, as depicted in FIG. 20, the full engagement of the BFS vial 210 with the connector 250, e.g., in the third or activated state, while forming the opening at "C", may form a seal at "D", e.g., to prevent leakage of the fluid backward (retrograde) into the BFS port 250-1. As the BFS vial 210 continues to be threaded into and accordingly axially advance into the BFS port 250-1, for example, the fluid seal 214 may be engaged with the seal flange 254 of the connector 250. The seal flange 254 may comprise a harder plastic than the fluid seal 214, for example, and the fluid seal 214 may accordingly deform to form an annular depression or seal at "D" in response to being engaged with the seal flange 254. This annular depression, which the seal flange 254 creates and in which the seal flange 254 becomes disposed, may act as a 0-ring or seal that prevents fluid leakage outside of the desired fluid pathway toward (antegrade) the needle 280.

According to some embodiments, application of a reverse axial force or pressure, e.g., imparted by an attempt to reinflate or reform the compressible reservoir 220, may be applied to reintroduce the fluid (the same fluid that was expelled and/or a different fluid) into the needle 280. Such reverse force/pressure may permit some fluid to re-enter the valve seat 250-2 but may also urge the tear-activation valve 230 toward the BFS vial 210 to block or re-seal the opening "C" (not shown). In some embodiments, in the case that the fluid seal 214 was originally compromised at the juncture of the fluid seal 214 and the tear-activation valve 230 (e.g., at "C"), pressure (toward the BFS vial 210 as applied by the fluid and/or suction force applied by the BFS vial 210) may cause the tear- activation valve 230 (or a portion thereof) to enter or plug the hole/opening "C", thereby preventing the fluid from entering/re-entering the BFS vial 210. In such a manner, for example, the existence of the tear-activation valve 230 between the BFS vial 210 and the needle 280 (e.g., as disposed or "trapped" in the valve seat 250-2) may comprise an AD device that becomes active at the same time that the pre-filled medical delivery assembly 200 is activated for use (e.g., upon achieving the third state by fully-engaging/coupling the BFS vial 210 with the administration assembly 240 and thereby causing the tear-activation valve 230 to form the opening "C"). In some embodiments, an additional one-way valve (not shown; e.g., a float and/or "bullet" valve) may be disposed in the fluid path (e.g., within a valve chamber formed in or by the connector 250; not shown) between the BFS vial 210 and the needle 280 to further prevent re-filling or re-use of the pre-filled medical delivery assembly 200 (and/or components thereof such as the BFS vial 210 and/or the administration assembly 240).

In some embodiments, after achieving the third or activated state in which the tear-activation valve 230 is engaged with the shear projection(s) 260 to produce the opening "C", removal or disengagement of the BFS vial 210 from the connector 250 may prevent re-use of the pre-filled medical delivery assembly 200 (e.g., defining an AD device). As shown in FIG. 2P, for example, in the case that the BFS vial 210 is removed from the BFS port 250- 1, the tear-activation valve 230 may remain lodged in the valve seat 250-2. Whether originally fully separated from the BFS vial 210 upon activation or whether separation is completed upon removal of the BFS vial 210, for example, the interference of the ears 230-2 with the valve seat 250-2 may cause the tear-activation valve 230 to remain in the valve seat 250-2. As depicted in FIG. 2Q, in the case that a new BFS vial 210ii comprising a new tear-activation valve 230ii is threaded into the BFS port 250-1, the new tear-activation valve 230ii may be prevented from entering the valve seat 250-2 by the presence of the original tear-activation valve 230. Particularly in the case where the BFS vial 210 is sized to deliver between one and ten milliliters (Iml - 10ml) of fluid, the size of the tear-activation valve 230 and the valve seat 250-2 is so small that a user would need specialized tools (such as a needle) to be able to remove/unseat the tear-activation valve 230.

In some embodiments, the needle 280 may comprise a needle shaped and/or sized for at least one of subcutaneous, intramuscular, intradermal, and intravenous injection of the fluid agent into the patient. For ease of explanation and description, the figures and the description herein generally refer to the needle 280 as a needle. However, it should be noted that, in other embodiments, the needle 280 may also or alternatively comprise a nozzle (not shown) configured to control administration of the fluid agent to the patient. The nozzle may comprise a spray nozzle, for example, configured to facilitate dispersion of the fluid agent into a spray. Accordingly, a version of the administration assembly 240 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a version of the administration assembly 240 fitted with a droplet nozzle may be useful in the administration of a fluid agent by way of droplets, such as administration to the eyes, topical administration, and the like.

The fluid or drug agent may include any type of agent to be injected into a patient (e.g., mammal, either human or non-human) and capable of producing an effect (alone, or in combination with an active ingredient). Accordingly, the agent may include, but is not limited to, a vaccine, a drug, a therapeutic agent, a medicament, a diluent, an active ingredient, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components such as the BFS vial 210 and/or the administration assembly 240.

According to some embodiments, the administration assembly 240 may be composed of a medical grade material. In some embodiments, the administration assembly 240 may be composed of a thermoplastic polymer or other "hard" plastic (e.g., greater than eighty (80) on the Rockwell "R" scale), including, but not limited to, polybenzimidazole, ABS, polystyrene, polyvinyl chloride, or the like. In some embodiments, the pre-filled medical delivery assembly 200 may be advantageously manufactured (in mass quantities) in separate parts or portions, namely, at least the "soft" plastic BFS vial 210 portion (e.g., a "first" piece) and the "hard" plastic administration assembly 240 (e.g., the "second" piece), with such different plastic parts/portions being selectively coupled to administer a medication to a patient.

In some embodiments, fewer or more components 210, 210ii, 212, 214, 216, 218, 220, 230, 230ii, 230-1, 230-2, 234, 240, 250, 250-1, 250-2, 250-3, 250-4, 250-5, 250-6, 252, 254, 256, 258, 258-1, 258-2, 260, 260-1, 270, 270-1, 272, 280, 280-1, 290 and/or various configurations of the depicted components 210, 210ii, 212, 214, 216, 218, 220, 230, 230ii, 230-1, 230-2, 234, 240, 250, 250-1, 250-2, 250-3, 250-4, 250-5, 250-6, 252, 254, 256, 258, 258-1, 258-2, 260, 260-1, 270, 270-1, 272, 280, 280-1, 290may be included in the pre-filled medical delivery assembly 200 without deviating from the scope of embodiments described herein. In some embodiments, the components 210, 210ii, 212, 214, 216, 218, 220, 230, 230ii, 230-1, 230-2, 234, 240, 250, 250-1, 250-2, 250-3, 250- 4, 250-5, 250-6, 252, 254, 256, 258, 258-1, 258-2, 260, 260-1, 270, 270-1, 272, 280, 280-1, 290may be similar in configuration and/or functionality to similarly named and/or numbered components as described herein. According to some embodiments, the pre-filled medical delivery assembly 200 may comprise only the BFS vial 210, only the administration assembly 240, or only the connector 250.

### III. Pre-filled BFS Vials with Tear-Activation Valves

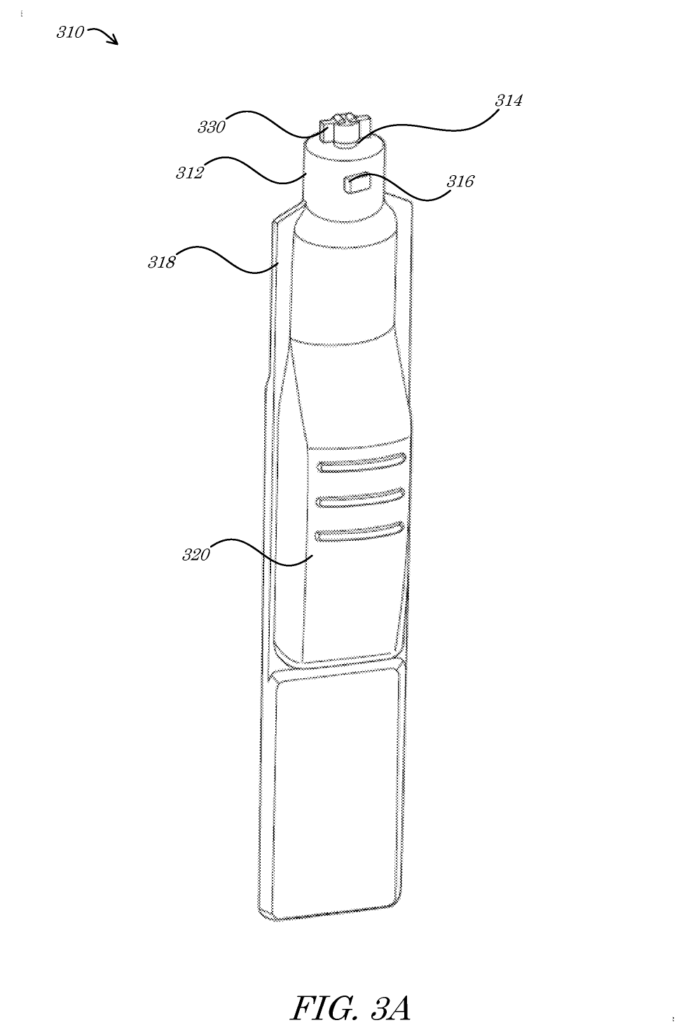

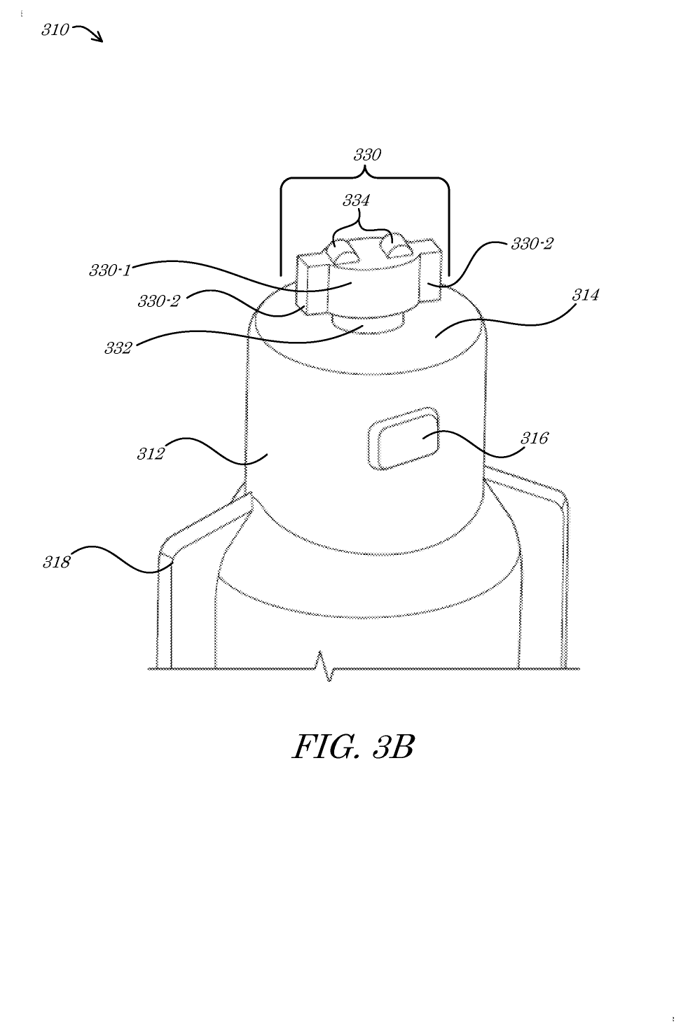

Referring to FIG. 3A, FIG. 3B, FIG. 3C, FIG. 3D, FIG. 3E, FIG. 3F, FIG. 3G, and FIG. 3H, various views of a pre-filled medical BFS vial 310 according to some embodiments are shown. The pre-filled medical BFS vial 310 may comprise, for example, a BFS bottle, vial, and/or container manufactured via a BFS process in which a fluid (not separately depicted) is injected into the pre-filled medical BFS vial 310 during the manufacturing process (e.g., in a sterile environment). According to some embodiments, the pre-filled medical BFS vial 310 may comprise and/or define a neck portion 312 that terminates at a fluid seal 314 disposed at a first end of the pre-filled medical BFS vial 310. In some embodiments, the neck portion 312 may comprise and/or define one or more mounting flanges 316 formed as angled radial exterior flanges or projections, e.g., the exemplary opposed rounded parallelogram- shaped mounting flanges 316 as depicted. According to some embodiments, the pre-filled medical BFS vial 310 may comprise a side flange 318 which may, for example, comprise unmolded portions of fused parison. In some embodiments, the pre-filled medical BFS vial 310 may comprise and/or define a compressible reservoir 320 storing the fluid (or one or more fluids such as both air and a dose of liquid medicament).

In some embodiments, various features of the of the pre-filled medical BFS vial 310 may provide distinct advantages with respect to previous BFS designs, particularly with respect to previous twist-to-open designs. In the case of the neck portion 312, for example, a cylindrical shape (as depicted) may provide for enhanced strength and/or ease of insertion for mating with an administration assembly (not shown; e.g., the administration assembly 140 of FIG. 1B, FIG. 1C, and/or FIG. 1K herein). According to some embodiments, the shape and/or dimensions of the mounting flange(s) 316 may enable the pre-filled medical BFS vial 310 to be readily and/or securely coupled to an administration assembly such as an injection-enabled module. The mounting flange(s) 316 may, for example, provide guideposts or keys that enable the pre-filled medical BFS vial 310 to be indexed, keyed, and/or mated with threads of an administration assembly, without requiring corresponding threads on the pre-filled medical BFS vial 310. In some embodiments, the pre-filled medical BFS vial 310 may also or alternatively comprise and/or define a tear-activation valve 330 that provides functionality not available in previous twist-to-open or tear-to-open vials.

According to some embodiments for example, the tear-activation valve 330 may be formed at the first end of the pre-filled medical BFS vial 310, e.g., at or on the fluid seal 314. In some embodiments, the tear-activation valve 330 may be formed with and/or otherwise comprise a geometry that is not readily engaged by human hands or fingers, but that is instead configured for mechanical shearing to permit opening of the pre-filled medical BFS vial 310. In the case that the pre-filled medical BFS vial 310 is sized to contain (e.g., within the compressible reservoir 320) less than ten milliliters (10-ml) of fluid, for example, the tear-activation valve 330 may comprise dimensions between about five and twenty millimeters (5 mm to 20 mm), which would be too small for a human to effectively engage with, e.g., to manually tear-off the tear-activation valve 330 and open the pre-filled medical BFS vial 310. According to some embodiments, the tear-activation valve 330 may comprise a diameter that is smaller than a diameter of the neck 312, the compressible reservoir 320, and/or the pre-filled medical BFS vial 310. In such a manner, for example, the tear-activation valve 330 may be sized to discourage human/tactile engagement while permitting the tear-activation valve 330 to fit within a cooperatively sized and/or shaped administration assembly/connector in which mechanical shearing and/or removal may be achieved.

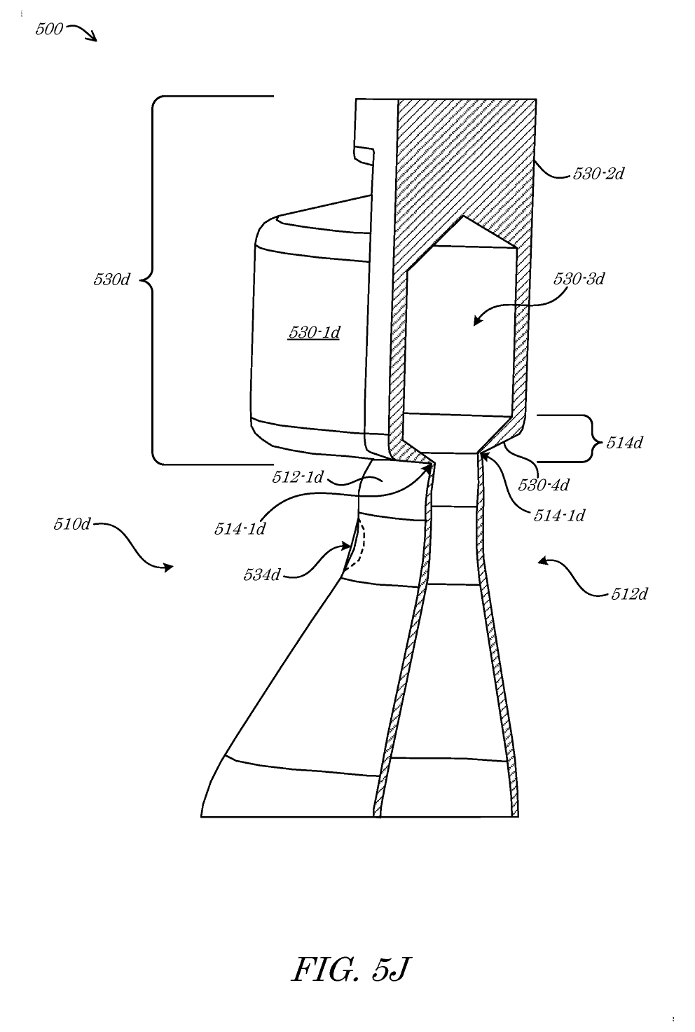

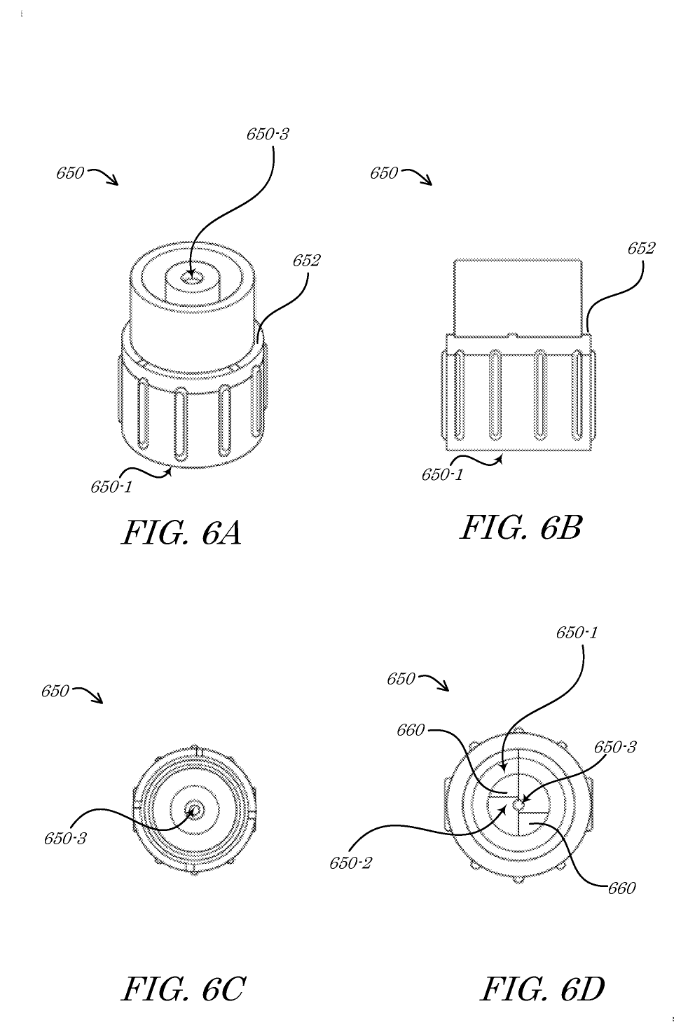

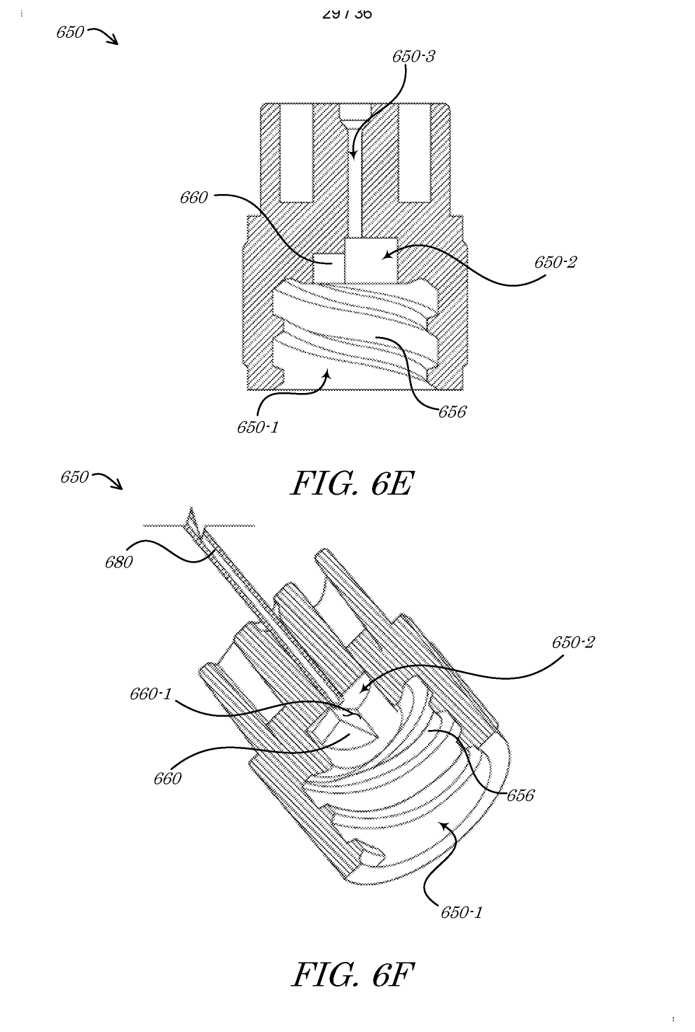

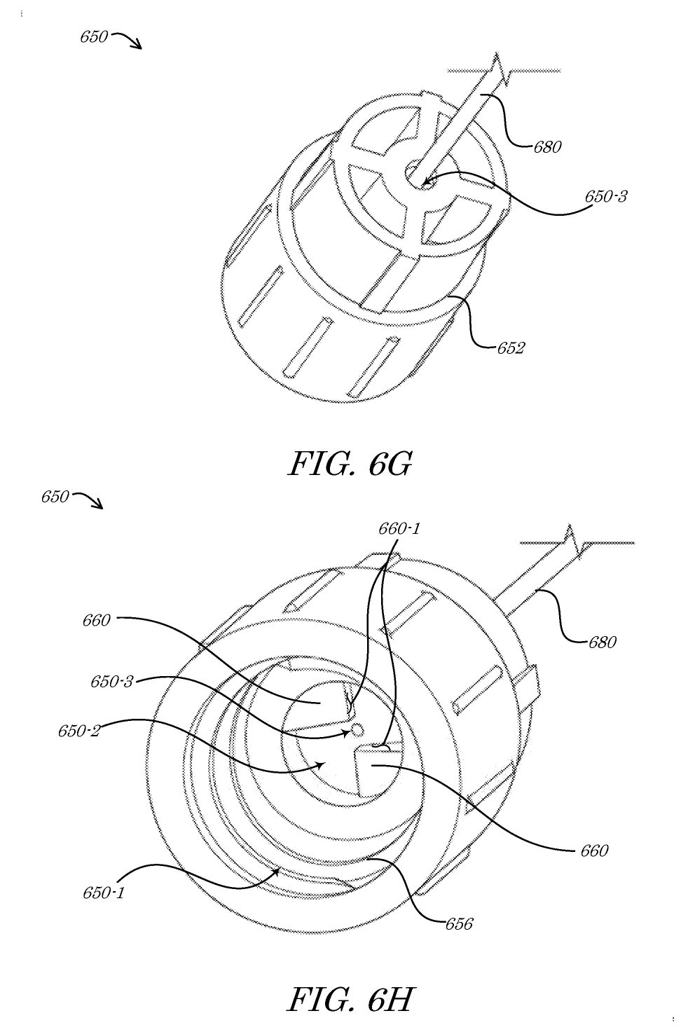

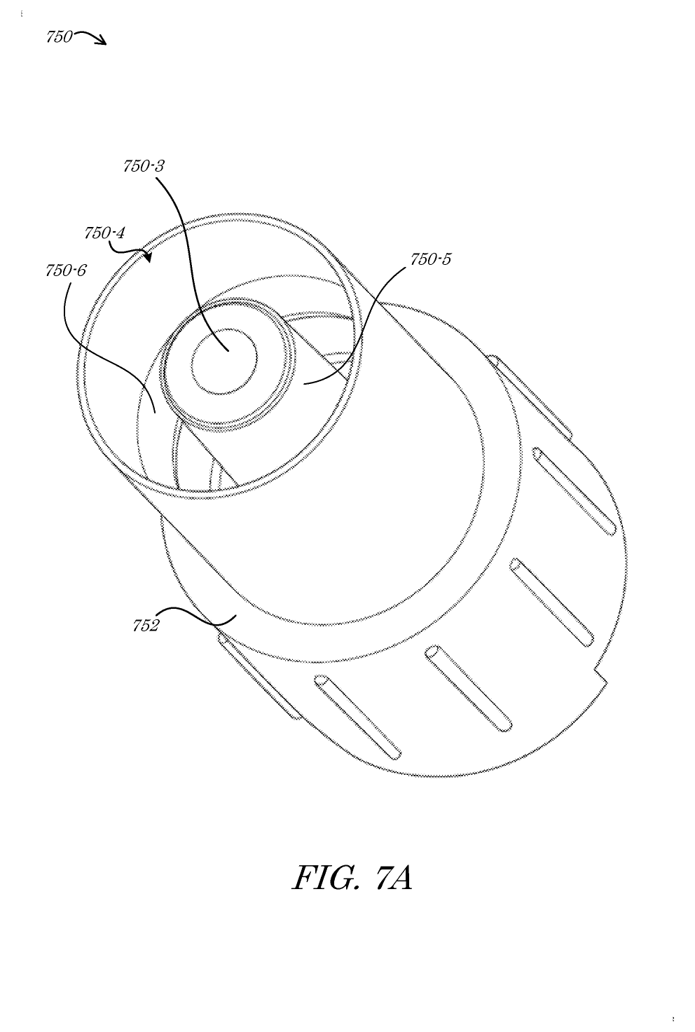

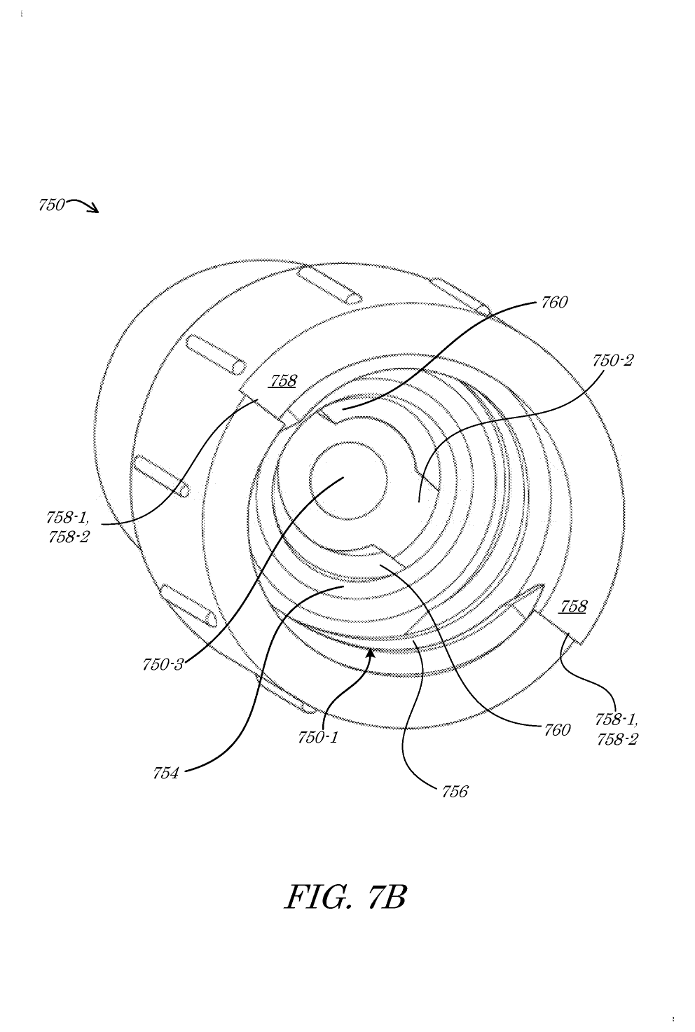

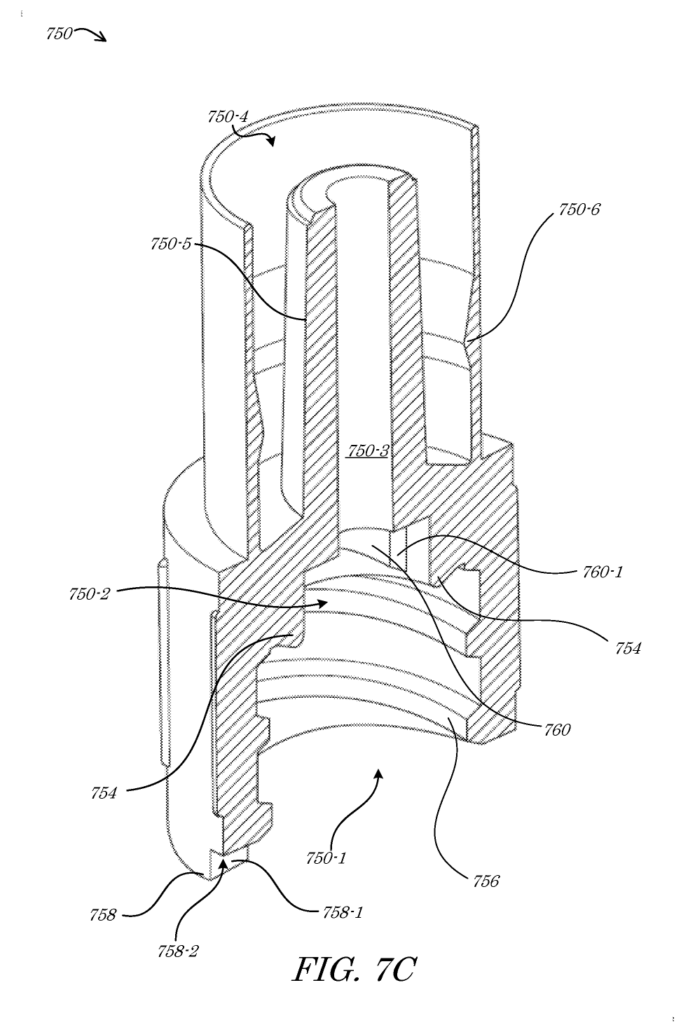

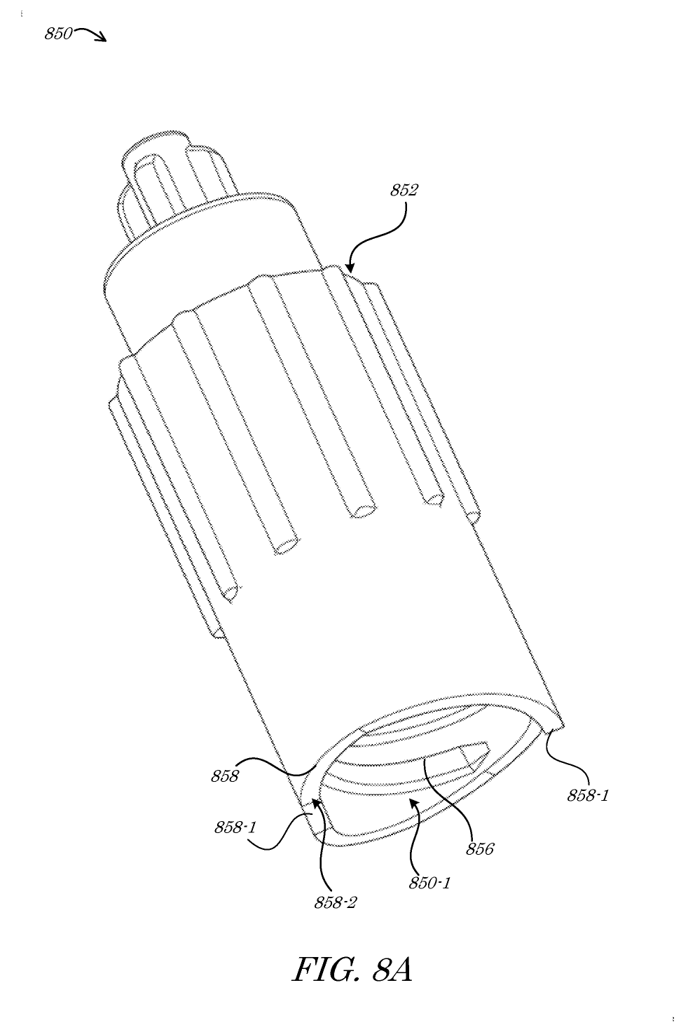

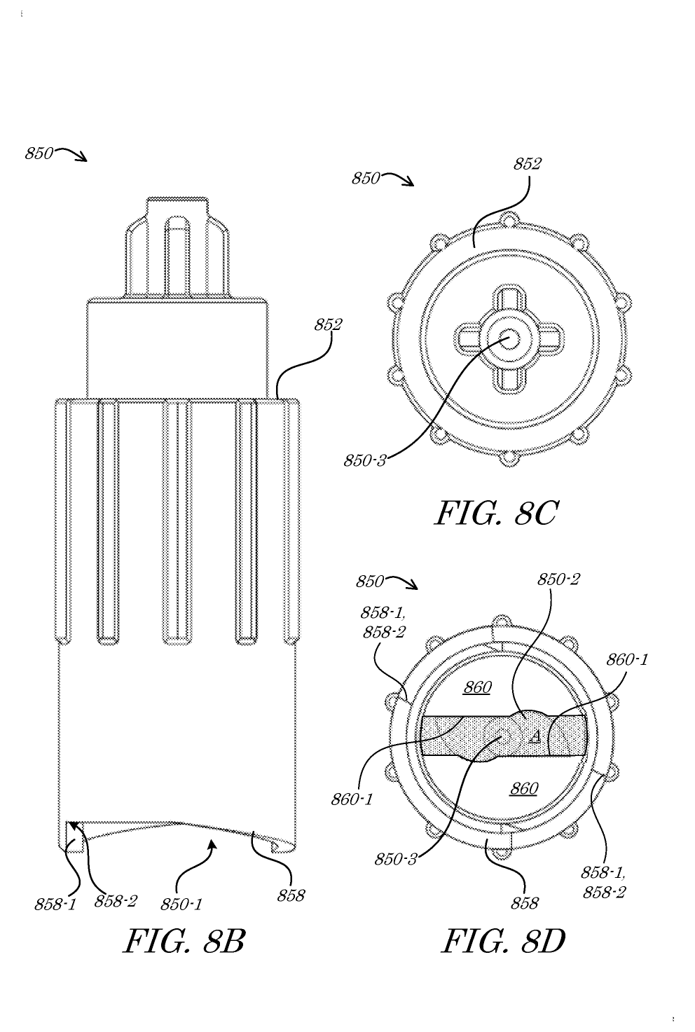

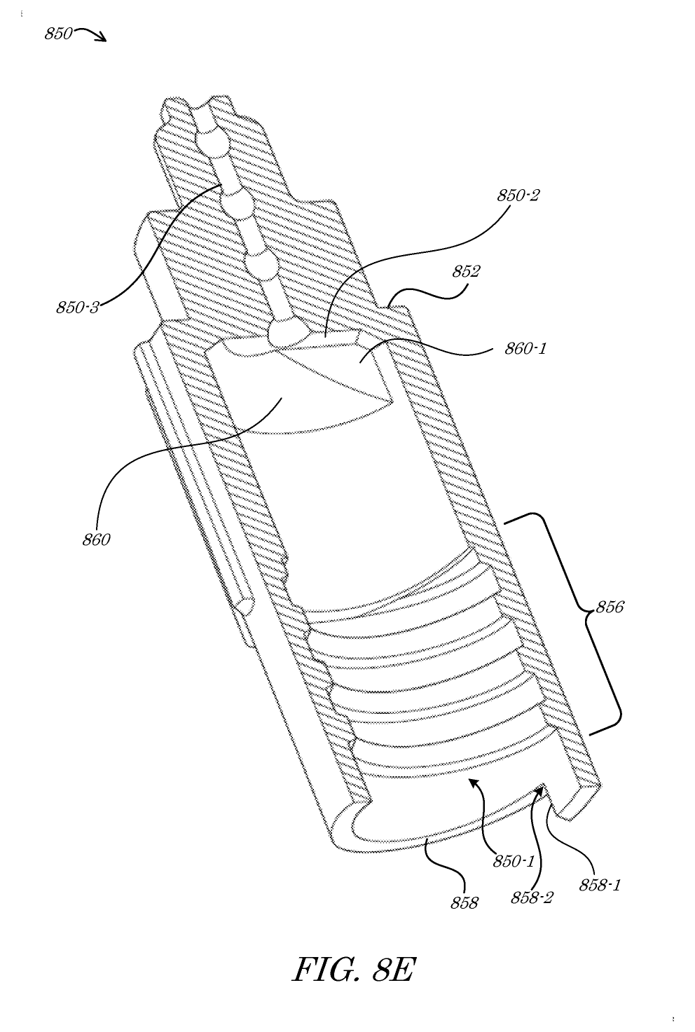

In some embodiments, the tear-activation valve 330 is instead sized and/or configured to facilitate mechanical removal when inserted into and engaged with a tear-activation device (not shown; e.g., the shear projections 160, 260, 660, 760, 860 and/or valve seats 150-2, 250-2, 650-2, 750-2, 850-2 of FIG. 1E, FIG. 1F, FIG. 1G, FIG. 1H, FIG. 11, FIG. 1J, FIG. 2E, FIG. 2G, FIG. 2J, FIG. 2K, FIG. 2L, FIG. 2M, FIG. 2N, FIG. 20, FIG. 2P, FIG. 2Q, FIG. 6D, FIG. 6E, FIG. 6F, FIG. 6H, FIG. 7B, FIG. 7C, FIG, 8D, and/or FIG. 8E herein). In the case that the tear-activation device is disposed in a sterile environment such as within a sealed or previously sealed administration assembly port (not shown), for example, the mechanical removal/compromising of the tear-activation valve 330 provides a cleaner and more reliable mechanism than manual human-hand opening of the pre-filled medical BFS vial 310. According to some embodiments, the tear-activation valve 330 may comprise an overall radial dimension that is less than a diameter of the neck 312 of the pre-filled medical BFS vial 310. In some embodiments, the tear-activation valve 330 may comprise and/or define a primary or first shaped portion 330-1 and/or a secondary or second shaped portion 330-2. With reference to FIG. 3B, for example, the first shaped portion 330-1 may comprise a circular or oval/elliptic cross-sectional shape to define a cylindrical body that extends from a post 332 at a proximal end to a terminus at a distal end. In some embodiments, the first shaped portion 330-1 may be sized and/or shaped to fit within a valve seat (e.g., cylindrical; not shown; e.g., the valve seat 150-2, 250- 2, 650-2, 750-2, 850-2 of FIG. 1E, FIG. 1F, FIG. 1G, FIG. 1H, FIG. 11, FIG. 1J, FIG. 2E, FIG. 2G, FIG. 2J, FIG. 2K, FIG. 2L, FIG. 2M, FIG. 2N, FIG. 20, FIG. 2P, FIG. 2Q, FIG. 6D, FIG. 6E, FIG. 6F, FIG. 6H, FIG. 7B, FIG. 7C, FIG,8D, and/or FIG. 8E herein) of an administration assembly. 8D, and/or FIG. 8E herein) of an administration assembly.

According to some embodiments, the second shaped portion 330-2 may comprise one or more rectangular or block projections that emanate radially outward from the first shaped portion 330-1. As depicted for purposes of non-limiting example in FIG. 3A, FIG. 3B, FIG. 3C, FIG. 3D, FIG. 3F, FIG. 3G, and FIG. 3H, for example, the first shaped portion 330-1 may comprise an elliptic cylinder and the second shaped portion 330-2 may comprise a pair of opposing rectangular cuboid or rectangular prism projections emanating radially outward from opposing sides of the first shaped portion 330-1. In some embodiments, the radial length or diameter of the first shaped portion 330- 1 may be sized to fit within an area of a valve seat that is free of projections/interference (e.g., a first or smallest diameter of the seat), while the total radial length or diameter of the tear-activation valve 330 (the length/diameter of the first shaped portion 330-1 plus the length/diameter of the second shaped portion 330-2) may be sized to fit within an overall or maximum extent of the valve seat (e.g., a second or largest diameter of the seat). According to some embodiments, the difference between the first and second seat diameters may equate to an amount of interference or projection of a shear projection, radially inward into the area or volume of the valve seat. In such a manner, for example, the first shaped portion 330-1 may rotate freely within the valve seat without obstruction, while the second shaped portion(s) 330-2 may cause the tear-activation valve 330 to engage with the shear projection in the case that the tear-activation valve 330 is rotated such that the second shaped portion(s) 330-2 engages with a surface of the shear projection. The tear-activation valve 330 may accordingly, in some embodiments, rotate freely throughout a first arc of rotation within the valve seat but may cause an interference or engagement between the second shaped portion(s) 330-2 and a shear projection throughout a second arc of rotation within the valve seat. According to some embodiments, threads of the administration assembly may be configured in conjunction with the valve seat such that full (or near-full) advancement of the mounting flange(s) 316 with the threads causes the second shaped portion(s) 330-2 to enter and/or traverse the second arc of rotation (e.g. and thereby engage with the shear projection(s)).

In some embodiments, the pre-filled medical BFS vial 310, the fluid seal 314, and/or the tear-activation valve 330 may comprise a shear element or post 332, e.g., extending between the proximal end of the first shaped portion 330-1 (or the tear-activation valve 330) and the fluid seal 314. According to some embodiments, the post 332 may comprise a cylindrical extension of the pre-filled medical BFS vial 310 that comprises and/or defines a diameter that is less than any diameter of the tear-activation valve 330. The smaller diameter of the post 332 may, for example, cause forces (e.g., rotational forces) exerted upon and/or experienced by the tear-activation valve 330 (e.g., due to engagement with the shear projection(s)) to be focused on a smaller area - i.e., an area of contact and/or connection between the post 332 and the fluid seal 314. In such a manner, for example, the post 332 may be configured to shear from the fluid seal 314 (partially or fully) upon full engagement or mating of the pre-filled medical BFS vial 310 with the administration assembly. In some embodiments, the thickness of the pre-filled medical BFS vial 310 at the juncture of the fluid seal 314 and the post 332 may be configured as a tear-line that is thinner than other areas (e.g., walls) of the pre-filled medical BFS vial 310, e.g., to focus material failure (e.g., due to externally applied forces) to occur at the juncture.

According to some embodiments, the tear-activation valve 330 may comprise and/or define one or more standoffs 334 formed, coupled, and/or otherwise emanating axially from the distal end, e.g., of the first shaped portion 330-1. The standoffs 334 may, for example, permit a fluid flow area or free space to be maintained between the tear-activation valve 330 and an interior surface of the administration assembly that may abut the distal end of the tear-activation valve 330. In such a manner, for example, in the case that an opening is created by a shearing of the post 332 from the fluid seal 314, fluid may flow from the opening, around the tear-activation valve 330 (e.g., between the first shaped portion 330-1 and an outer radial extent of a valve seat in which the first shaped portion 330-1 is disposed), through the space provided by the standoffs 334, and/or, e.g., through an administration member of the administration assembly.

In some embodiments, the post 332 may comprise a solid structure formed with or as part of the tear- activation valve 330 (and/or with or as part of the fluid seal 314). According to some embodiments, the post 332 may be hollow or define a bore 336 that is in fluid communication with the interior of the pre-filled medical BFS vial 310 (e.g., the interior of the neck 312 and/or the compressible reservoir 320). As depicted in FIG. 3G, for example, the bore 336 may extend from an interior volume of the pre-filled medical BFS vial 310, through the post 332, and/or into the first shaped portion 330-1 of the tear-activation valve 330. In such a manner, for example, forces (e.g., rotational shear forces) applied to the tear-activation valve 330 (e.g., due to a threaded coupling of the pre-filled medical BFS vial 310 with an administration assembly) may cause: (i) the first shaped portion 330-1 of the tear- activation valve 330 to tear (e.g., fully or partially separate) from the post 332, (ii) a side-wall of the post 332 to rupture or tear, (iii) the post 332 to tear from the fluid seal 314, and/or (iv) the fluid seal 314 to tear or rupture.

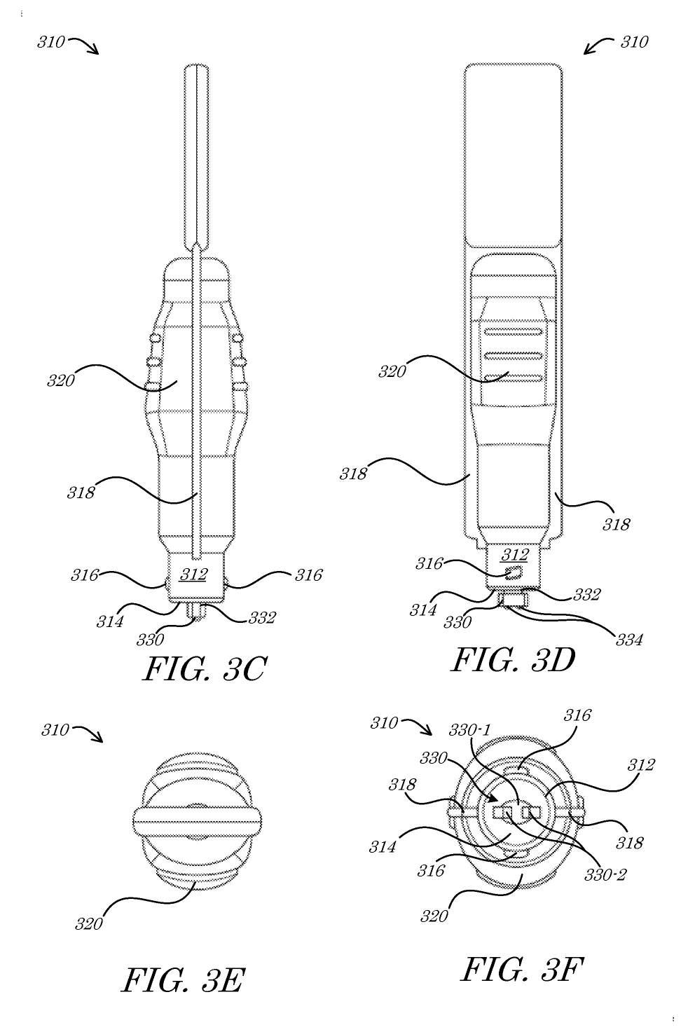

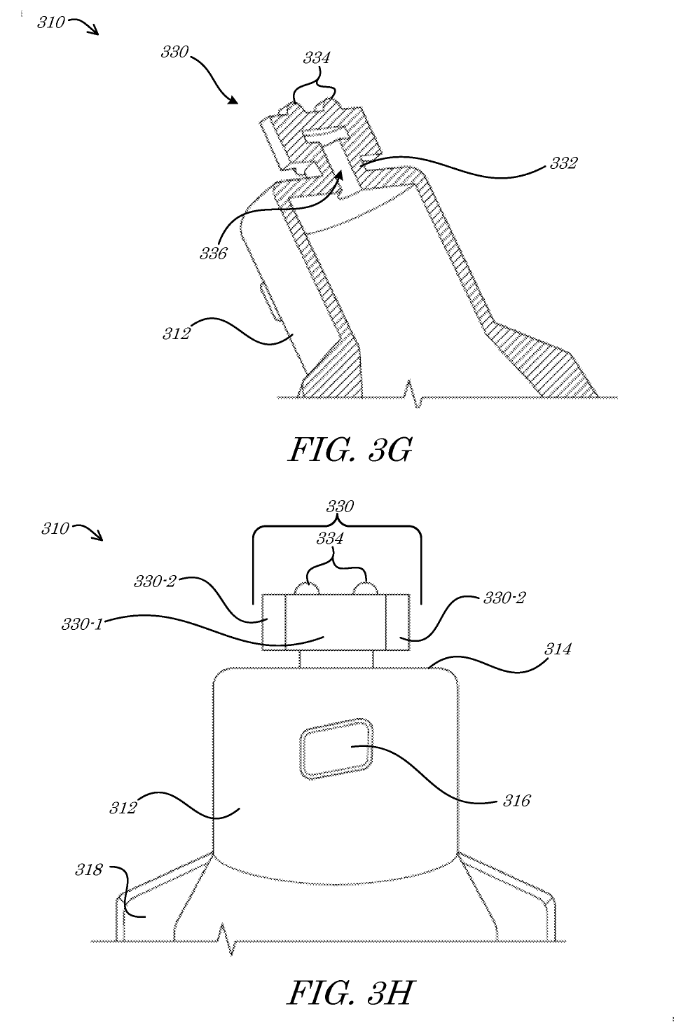

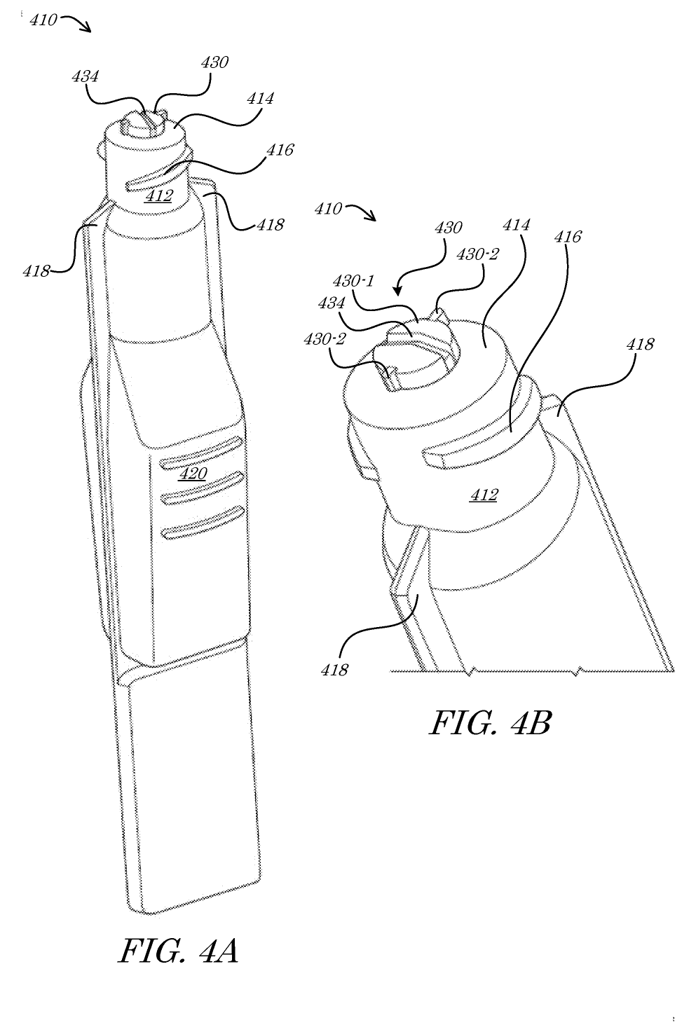

Referring to FIG. 4A and FIG. 4B, perspective views of a pre-filled medical BFS vial 410 according to some embodiments are shown. The pre-filled medical BFS vial 410 may comprise, for example, a BFS bottle, vial, and/or container manufactured via a BFS process in which a fluid (not separately depicted) is injected into the pre-filled medical BFS vial 410 during the manufacturing process (e.g., in a sterile environment). According to some embodiments, the pre-filled medical BFS vial 410 may comprise and/or define a neck portion 412 that terminates at a fluid seal 414 disposed at a first end of the pre-filled medical BFS vial 410. In some embodiments, the neck portion 412 may comprise and/or define one or more mounting flanges 416 formed as angled radial exterior flanges or projections, e.g., the example opposed angled tracks or flanges 416 as depicted. According to some embodiments, the pre-filled medical BFS vial 410 may comprise a side flange 418 which may, for example, comprise unmolded portions of fused parison. In some embodiments, the pre-filled medical BFS vial 410 may comprise and/or define a compressible reservoir 420 storing the fluid (or one or more fluids such as both air and a dose of liquid medicament).

In some embodiments, various features of the of the pre-filled medical BFS vial 410 may provide distinct advantages with respect to previous BFS designs, particularly with respect to previous twist-to-open designs. In the case of the neck portion 412, for example, a cylindrical shape (as depicted) may provide for enhanced strength and/or ease of insertion for mating with an administration assembly (not shown; e.g., the administration assembly 140, 240 of FIG. 1B, FIG. 1C, FIG. 1K, and/or FIG. 2A herein). According to some embodiments, the shape and/or dimensions of the mounting flange(s) 416 may enable the pre-filled medical BFS vial 410 to be readily and/or securely coupled to an administration assembly such as an injection-enabled module. The mounting flange(s) 416 may, for example, provide tracks or keys that enable the pre-filled medical BFS vial 410 to be indexed, keyed, and/or mated with threads of an administration assembly, without requiring corresponding threads on the pre-filled medical BFS vial 410. In some embodiments, the pre-filled medical BFS vial 410 may also or alternatively comprise and/or define a tear-activation valve 430 that provides functionality not available in previous twist-to-open or tear- to-open vials.

According to some embodiments for example, the tear-activation valve 430 may be formed at the first end of the pre-filled medical BFS vial 410, e.g., at or on the fluid seal 414. In some embodiments, the tear-activation valve 430 may be formed with and/or otherwise comprise a geometry that is not readily engaged by human hands or fingers, but that is instead configured for mechanical shearing to permit opening of the pre-filled medical BFS vial 410. In the case that the pre-filled medical BFS vial 410 is sized to contain (e.g., within the compressible reservoir 420) less than ten milliliters (10-ml) of fluid, for example, the tear-activation valve 430 may comprise dimensions between about five and twenty millimeters (5 mm to 20 mm), which would be too small for a human to effectively engage with, e.g., to manually tear-off the tear-activation valve 430 and open the pre-filled medical BFS vial 410.

In some embodiments, the tear-activation valve 430 is instead sized and/or configured to facilitate mechanical removal when inserted into and engaged with a tear-activation device (not shown; e.g., the shear projections 160, 260, 660, 760, 860 and/or valve seats 150-2, 250-2, 650-2, 750-2, 850-2 of FIG. 1E, FIG. 1F, FIG. 1G, FIG. 1H, FIG. 11, FIG. 1J, FIG. 2E, FIG. 2G, FIG. 2J, FIG. 2K, FIG. 2L, FIG. 2M, FIG. 2N, FIG. 20, FIG. 2P, FIG. 2Q, FIG. 6D, FIG. 6E, FIG. 6F, FIG. 6H, FIG. 7B, FIG. 7C, FIG. 8D, and/or FIG. 8E herein). In the case that the tear-activation device is disposed in a sterile environment such as within a sealed or previously sealed administration assembly port (not shown), for example, the mechanical removal/compromising of the tear-activation valve 430 provides a cleaner and more reliable mechanism than manual human-hand opening of the pre-filled medical BFS vial 410. According to some embodiments, the tear-activation valve 430 may comprise an overall radial dimension that is less than a diameter of the neck 412 of the pre-filled medical BFS vial 410. In some embodiments, the tear-activation valve 430 may comprise and/or define a primary or first shaped portion 430-1 and/or a secondary or second shaped portion 430-2. With reference to FIG. 4B, for example, the first shaped portion 430-1 may comprise a circular cross-sectional shape that extends from the fluid seal 414 at a proximal end to a terminus at a distal end. In some embodiments, the first shaped portion 430-1 may be sized and/or shaped to fit within a valve seat (e.g., cylindrical; not shown; e.g., the valve seat 150-2, 250-2, 650-2, 750-2, 850-2 of FIG. 1E, FIG. 1F, FIG. 1G, FIG. 1H, FIG. 11, FIG. 1J, FIG. 2E, FIG. 2G, FIG. 2J, FIG. 2K, FIG. 2L, FIG. 2M, FIG. 2N, FIG. 20, FIG. 2P, FIG. 2Q, FIG. 6D, FIG. 6E, FIG. 6F, FIG. 6H, FIG. 7B, FIG. 7C, FIG. 8D, and/or FIG. 8E herein) of an administration assembly.

According to some embodiments, the second shaped portion 430-2 may comprise one or more projections that emanate radially outward from the first shaped portion 430-1. As depicted for purposes of non-limiting example in FIG. 4A and FIG. 4B for example, the first shaped portion 430-1 may comprise a circular nub and the second shaped portion 430-2 may comprise a pair of opposing "ear" projections emanating radially outward from opposing sides of the first shaped portion 430-1. In some embodiments, the radial length or diameter of the first shaped portion 430-1 may be sized to fit within an area of a valve seat that is free of projections/interference (e.g., a first or smallest diameter of the seat), while the total radial length or diameter of the tear-activation valve 430 (the length/diameter of the first shaped portion 430-1 plus the length/diameter of the second shaped portion 430-2) may be sized to fit within an overall or maximum extent of the valve seat (e.g., a second or largest diameter of the seat). According to some embodiments, the difference between the first and second seat diameters may equate to an amount of interference or projection of a shear projection, radially inward into the area or volume of the valve seat. In such a manner, for example, the first shaped portion 430-1 may rotate freely within the valve seat without obstruction, while the second shaped portion(s) 430-2 may cause the tear-activation valve 430 to engage with the shear projection in the case that the tear-activation valve 430 is rotated such that the second shaped portion(s) 430-2 engages with a surface of the shear projection. The tear-activation valve 430 may accordingly, in some embodiments, rotate freely throughout a first arc of rotation within the valve seat but may cause an interference or engagement between the second shaped portion(s) 430-2 and a shear projection throughout a second arc of rotation within the valve seat.

According to some embodiments, threads of the administration assembly may be configured in conjunction with the valve seat such that full (or near-full) advancement of the mounting flange(s) 416 with the threads causes the second shaped portion(s) 430-2 to enter and/or traverse the second arc of rotation (e.g. and thereby engage with the shear projection(s)). In some embodiments, the total radial length or diameter of the tear-activation valve 430 (the length/diameter of the first shaped portion 430-1 plus the length/diameter of the second shaped portion 430-2) may be sized to be slightly smaller than the overall or maximum extent of the valve seat, e.g., to produce an interference or friction fit between the second shaped portion(s) 430-2 and side walls of the valve seat. As described herein, such interference may cause a deformation of the second shaped portion(s) 430-2, thereby providing an elastic force fit that acts to retain the tear-activation valve 430 in the valve seat. According to some embodiments, the tear-activation valve 430 may comprise and/or define a channel 434 formed at the distal end, e.g., of the first shaped portion 430-1. The channel 434 may, for example, permit a fluid flow area or free space to be maintained between the tear-activation valve 430 and an interior surface of the administration assembly that may about the distal end of the tear-activation valve 430. In such a manner, for example, in the case that an opening is created by a shearing of the tear-activation valve 430 from the fluid seal 414, fluid may flow from the opening, around the tear-activation valve 430 (e.g., between the first shaped portion 430-1 and an outer radial extent of a valve seat in which the first shaped portion 430-1 is disposed), and/or through the channel 434, and then, e.g., through an administration member of the administration assembly.

According to some embodiments, fewer or more components 412, 414, 416, 418, 420, 430, 430-1, 430-2, 434 and/or various configurations of the depicted components 412, 414, 416, 418, 420, 430, 430-1, 430-2, 434 may be included in the pre-filled medical BFS vial 410 without deviating from the scope of embodiments described herein. In some embodiments, the components 412, 414, 416, 418, 420, 430, 430-1, 430-2, 434 may be similar in configuration and/or functionality to similarly named and/or numbered components as described herein. According to some embodiments, the pre-filled medical BFS vial 410 may comprise the tear-activation valve 430 but not the channel 434. In some embodiments, the pre-filled medical BFS vial 410 may comprise the tear-activation valve 430 but not the mounting flange 416 and/or the side flange 418. Although two opposing second shaped portion(s) 430- 2 of the tear-activation valve 430 are depicted for purposes of example, in some embodiments the tear-activation valve 430 of the pre-filled medical BFS vial 410 may comprise only a single second shaped portion 430-2 and/or the first shaped portion 430-1 may be shaped to engage with a shear projection without requiring a second shapedportion(s) 430-2

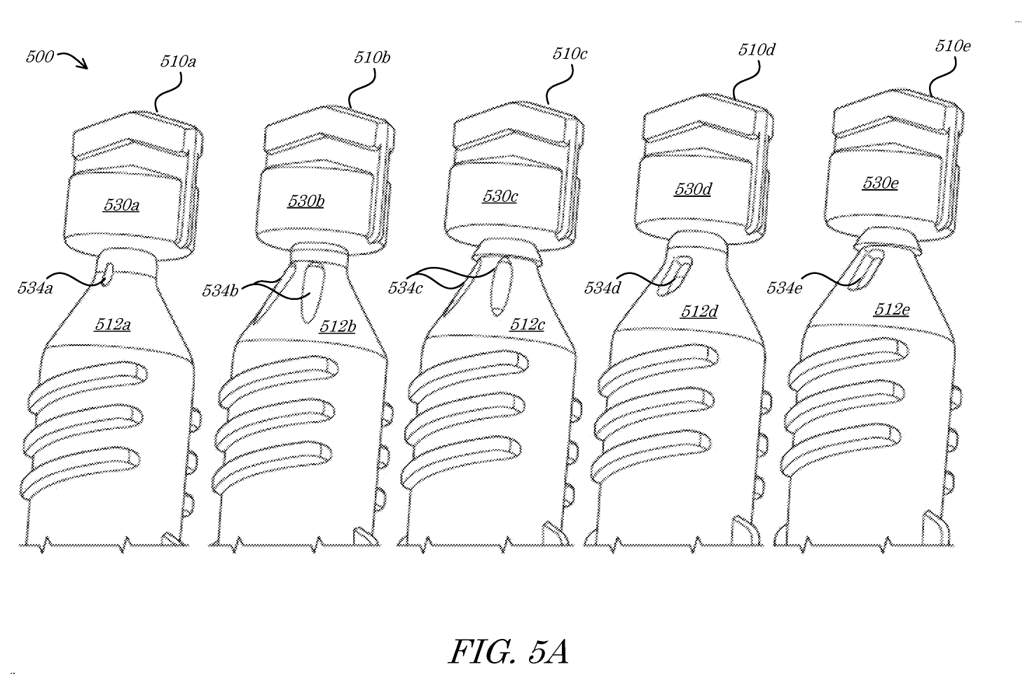

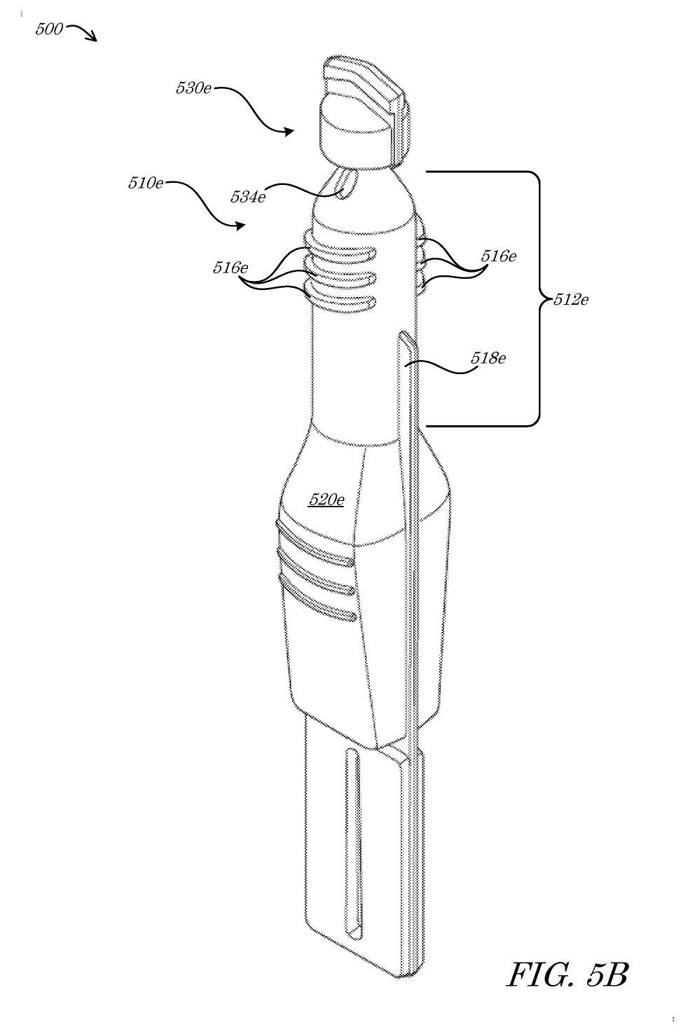

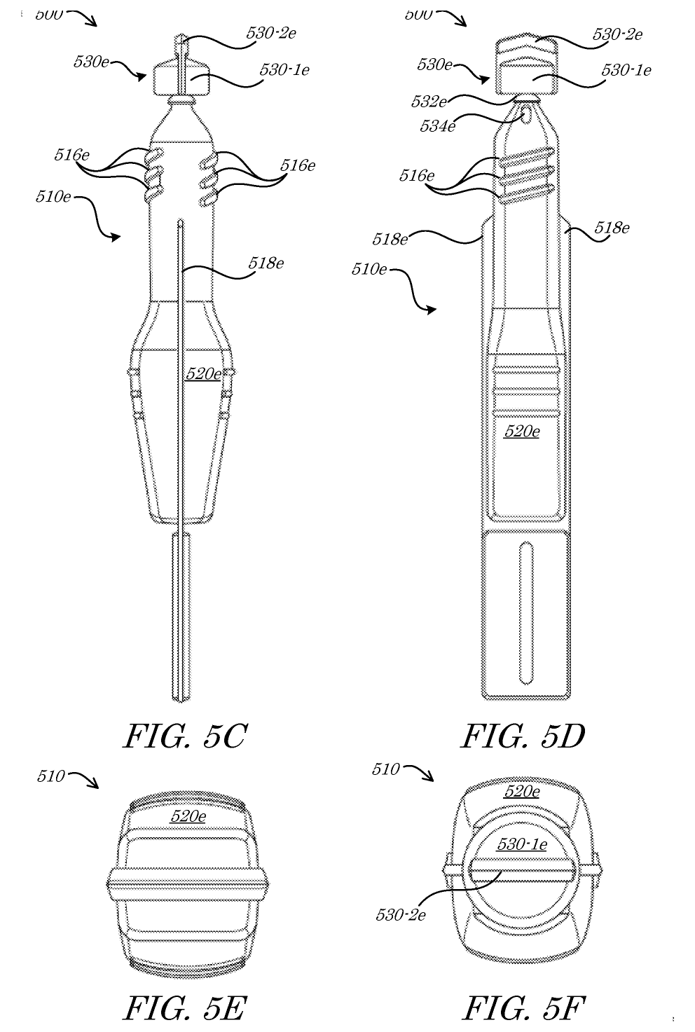

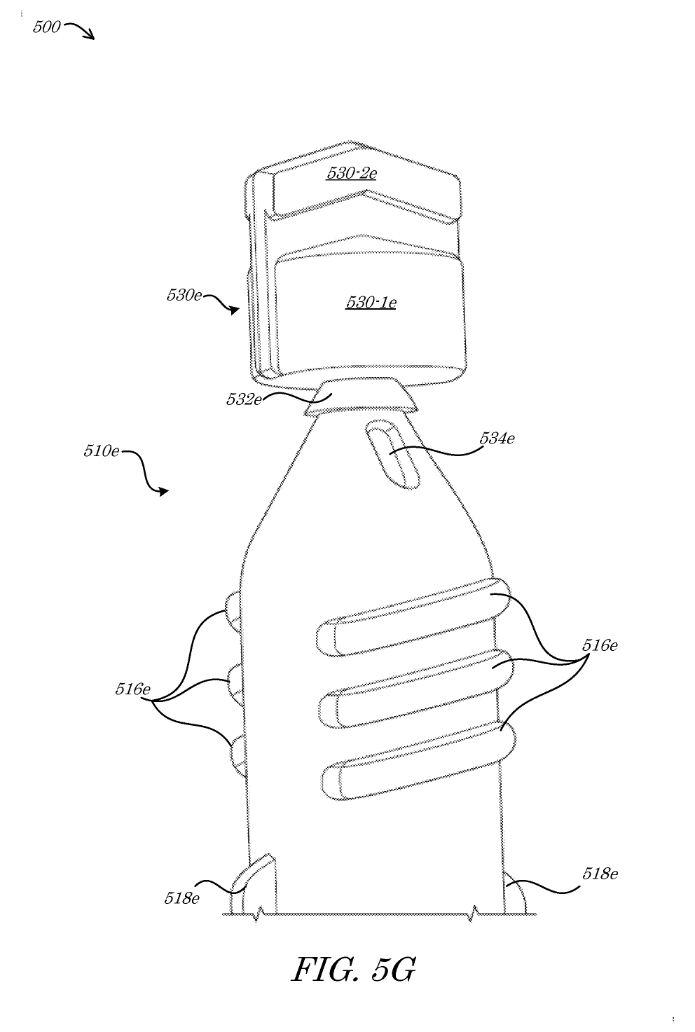

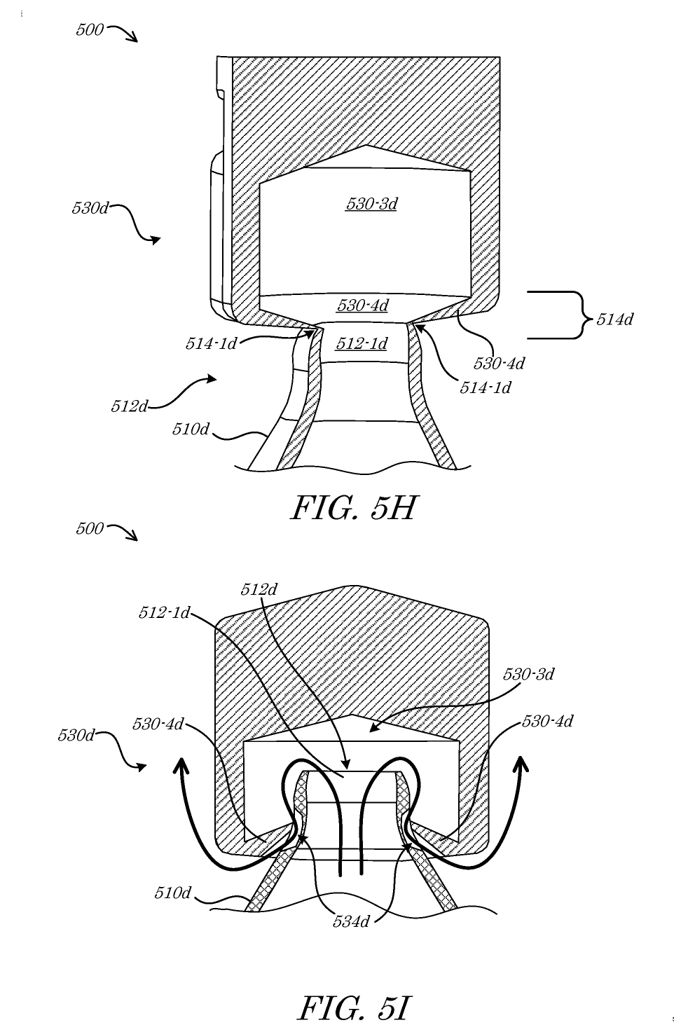

Turning to FIG. 5A, FIG. 5B, FIG. 5C, FIG. 5D, FIG. 5E, FIG. 5F, FIG. 5G, FIG. 5H, FIG. 51, and FIG. 5J, various views of a pre-filled BFS vial system 500 according to some embodiments are shown. In some embodiments, as shown in FIG. 5A, different configurations of BFS vials 510a-e may be included (separately or connected) in the pre-filled BFS vial system 500. According to some embodiments, the BFS vials 510a-e may comprise and/or define neck portions 512a-e (FIG. 5A, FIG. 52, FIG. 5H, and FIG. 5J) Proximate to a first end (e.g., a distal end) of the BFS vials 510a-e (and/or emanating from a body, not separately labeled, thereof). In some embodiments, as depicted in the cross-sectional views of FIG. 5H and FIG. 5J, one of (e.g., a fourth) the BFS vials 510d may comprise a neck 512d that defines and/or includes a tapered portion 512-1d where a wall thickness of the BFS vial 510d decreases toward a fluid seal 514d at a distal end of the neck 512d. In some embodiments, a junction of the fluid seal 514d with the neck 512d may comprise and/or define a pinch point 514-1d. The pinch point 514-1d may comprise and/or define, for example, a point, line, shape, plane, and/or area at which the BFS vial 510d is configured to fail (e.g., tear and/or breach). In some embodiments, and with reference to FIG. 5B, FIG. 5C, FIG. 5D, FIG. 5E, FIG. 5F, and FIG. 5G, one of (e.g., a fifth) the BFS vials 510e may comprise and/or define a plurality of angled mounting flanges 516e, one or more side flanges 518e, and/or a compressible reservoir 520e. According to some embodiments, the BFS vial 510e may comprise a tear-activation valve 530e formed, disposed, and/or coupled at a distal end of the neck 512e thereof. The tear-activation valve 530e may, for example, comprise a first shaped portion 530-1e and/or a second shaped portion 530-2e coupled to a post or flange 532e disposed at the distal end of the neck 512e. According to some embodiments, the fluid seal 514d may be formed at the junction between the tear-activation valve 530d (and/or first shaped portion 530-1d thereof) and the neck 512d. In some embodiments, the second shaped portion 530-2e may comprise and/or define a linear and/or rectangular shape extending along or across a diameter of the tear-activation valve 530e. According to some embodiments, the BFS vial 510e may comprise and/or define the channel 534e (FIG. 5A, FIG. 5B, FIG. 5D, and FIG. 5G), e.g., formed in or on the neck 512e. The channel 534e may comprise, for example, a portion of the neck 512e that comprises an exterior surface disposed at a different (smaller or larger) radial diameter than the surrounding exterior surface areas of the neck 512e. The channel 534e may comprise, for example, extend axially along a portion of the outer radial surface of the neck 512e, e.g., between the angled mounting flanges 516e and the fluid seal 514d. In some embodiments, one or more of the channels 534a-e such as first, second, and third channels 534a-c as-depicted, may comprise and/or define one or more ridges and/or axial projections, e.g., spaced to provide and/or define one or more valleys, detents, and/or channels therebetween.

According to some embodiments, the angled mounting flanges 516e may comprise a series (e.g., three (3), as depicted) of angled projections, detents, protrusions, and/or features that are equally spaced at different axial positions of the neck 512e. In some embodiments, the angled mounting flanges 516e may comprise circumferentially elongated features that extend between thirty and forty-five percent (30-45%) of the circumference of the neck 512e and/or are angled with respect to a normal plane (not shown) to the axis of the pre-filled BFS vial system 500 (and/or of the BFS vial 510e). In some embodiments, as depicted for purposes of non-limiting example, a plurality (e.g., two (2), as depicted) of sets of angled mounting flanges 516e may be disposed, formed, and/or defined about the circumference of the neck 512e. As depicted, the angled mounting flanges 516e may be disposed axially between the side flanges 518e and the tear-activation valve 530e (and/or the flange 532e and/or the channel 534e). In some embodiments, the flange 532e may comprise an axially tapered shaped portion of the neck 512e that, e.g., tapers from a first smaller diameter at a junction with the tear-activation valve 530e to a second larger diameter proximate to the channel 534e.

With reference to the one of (e.g., the fourth) the BFS vials 510d depicted in FIG. 5H, FIG. 51, and FIG. 5J, an advantageous configuration of the tear-activation valve 530d thereof may be understood. The neck 512d may comprise and/or define the tapered portion 512-1d, for example, where a wall thickness of the BFS vial 510d decreases toward the fluid seal 514d at the distal end of the neck 512d. In some embodiments, the tear-activation valve 530d may define a valve volume 530-3d formed within the first shaped portion 530-1d thereof. As depicted in FIG. 5H and FIG. 5J, for example, the first shaped portion 530-1d may comprise a hollow cylindrical portion of the BFS vial 510d formed at the distal end of the neck 512d (and/or defining a distal end of the BFS vial 510d) and defining the valve volume 530-3d therewithin. In some embodiments, the valve volume 530-3d may be in fluid communication with the interior of the BFS vial 510d (e.g., may be part of and/or contiguous with the interior volume of the BFS vial 510d). According to some embodiments, the tear-activation valve 530d may join and/or be coupled to the distal end of the neck 512d (and/or a distal end of the tapered portion 512-1d thereof), at and/or defining the fluid seal 514d.

The fluid seal 514d may, for example, comprise the pinch point 514-1d and/or the tapered portion 512-1d and/or a bottom surface 530-4d of the tear-activation valve 530d (and/or portions thereof). In some embodiments,

the bottom surface 530-4d of the tear-activation valve 530d (and/or of the first shaped portion 530-1d thereof) may comprise a tapered wall thickness that narrow from an outer diameter of the first shaped portion 530-1d to the pinch point 514-1d. In such a manner, for example, an application of force that imparts tension and/or compression between the neck 512d and the tear-activation valve 530d may cause the BFS vial 510d to fail, e.g., rupturing and/or exposing the interior volume, at (and/or proximate to) the pinch point 514-1d (e.g., along a circumference between the tapered portion 512-1d of the neck 512d and/or the bottom surface 530-4d of the tear-activation valve 530d). In some embodiments, the tearing of the fluid seal 514d may cause the tear-activation valve 530d to become partially or fully separated from the BFS vial 510d.

According to some embodiments, such as in the case that the fluid seal 514d is breached while the BFS vial 510d is coupled to a cooperatively-configured administration member (not shown; e.g., the administration assemblies 140, 240 of FIG. 1B, FIG. 1C, FIG. 1K, and/or FIG. 2A herein), the tear-activation valve 530d may be maintained adjacent to and/or engaged with the BFS vial 510d (e.g., even in the case that the tear-activation valve 530d is separated from the BFS vial 510d). In some embodiments, the tear-activation valve 530d may engaged with the neck 512d to of the BFS vial 510d (i) limit or govern the flow of fluid from the BFS vial 510d (e.g., outflow or effluent) and/or (ii) limit or prevent the introduction (and/or reintroduction) of fluid into the BFS vial 510d (e.g., inflow or influent). As shown in FIG. 51, for example, after separation of the tear-activation valve 530d from the BFS vial 510d, the bottom surface 530-4d of the tear-activation valve 530d may be engaged to accept a portion of the neck 512d (such as the tapered portion 512-1d). The bottom surface 530-4d may be deflected upwards (e.g., axially away from) the BFS vial 510d, for example, establishing a fit (e.g., an interference fit) between the newly formed opening of the first shaped portion 530-1d in the bottom surface 530-4d and an outer diameter of the neck 512d.

In some embodiments, the engagement of the tear-activation valve 530d with the BFS vial 510d may define a fluid pathway as depicted for purposes of non-limiting example with the bold, arrow-tipped lines in FIG. 51. As shown, the fluid from the BFS vial 510d may flow out from the top or distal end of the BFS vial 510d and/or the neck 512d (e.g., where the fluid seal 514d was breached), into the valve volume 530-3d, and out through the channels 534e. According to some embodiments, an attempt to refill the BFS vial 510d may cause the tear- activation valve 530d to more fully engage with (e.g., accept a larger axial extent of) the neck 512d, such that the channels 534e are substantially or entirely disconnected from fluid communication with any environment outside of the BFS vial 510d and the valve volume 530-3d. In such a manner, for example, the tear-activation valve 530d may (i) provide a sterile (e.g., inside of an administration assembly) mechanism to open or activate the BFS vial 510d (e.g., by enabling the fluid seal 514d to be breached in a sterile environment), (ii) enable fluid to be dispensed from the BFS vial 510d, and/or (iii) prevent the BFS vial 510d from being filled/re-filled. In some embodiments, such as in the case that the flange 532e is forced into the valve volume 530-3d, the flange 532e may impede attempts at removing the neck 512e from the valve volume 530-3d.

According to some embodiments, fewer or more components 510a-e, 512a-e, 512-1d, 514d, 514-1d, 516e, 518e, 520e, 530a-e, 530-1d, 530-1e,530-1e, 530-2d, 530-2e,530-2e, 530-3d, 530-4d, 532e, 534a-e and/or various configurations of the depicted components 510a-e, 512a-e, 512-1d, 514d, 514-1d, 516e, 518e, 520e, 530a-e, 530-1d, 530-le, 530-2d,530-2d, 530-2e, 530-3d,530-3d, 530-4d, 532e, 534a-e may be included in the pre-filled BFS vial system 500 without deviating from the scope of embodiments described herein. In some embodiments, the components 510a-e, 512a- e, 512-1d, 514d, 514-1d, 516e, 518e, 520e, 530a-e, 530-1d, 530-1e,530-1e, 530-2d, 530-2e,530-2e, 530-3d, 530-4d, 532e, 534a- e may be similar in configuration and/or functionality to similarly named and/or numbered components as described herein. According to some embodiments, the pre-filled BFS vial system 500 and/or one or more of the BFS vials 510a-e thereof may comprise the tear-activation valve 530a-e but not the channel(s) 534a-e. In some embodiments, the channel(s) 534a-e may comprise standoffs or projections instead of or in addition to detents or voids. According to some embodiments, the pre-filled BFS vial system 500 and/or one or more of the BFS vials 510a-e may comprise the tear-activation valve 530a-e but not the mounting flange(s) 516e and/or the side flange(s) 518e. In some embodiments the tear-activation valve 530a-e may be shaped to selectively engage with a shear projection (not shown) that imparts force to shear the tear-activation valve 530a-e from the BFS vials 510a-e. In some embodiments, the flange 532e may not be included in the pre-filled BFS vial system 500.

### IV. BFS CONNECTORS FOR USE WITH TEAR-ACTIVATION VALVE

Turning to FIG. 6A, FIG. 6B, FIG. 6C, FIG. 6D, FIG. 6E, FIG. 6F, FIG. 6G, and FIG. 6H, various views of a BFS connector 650 according to some embodiments are shown. In some embodiments, the BFS connector 650 may be a component of and/or comprise an administration assembly that is configured to mate with a BFS vial or a portion thereof (not shown in FIG. 6A, FIG. 6B, FIG. 6C, FIG. 6D, FIG. 6E, FIG. 6F, FIG. 6G, and FIG. 6H; e.g., the neck 112, 212, 312, 412, 512a-e of FIG. 1A, FIG. 1B, FIG. 1C, FIG. 1D, FIG. 1E, FIG. 1F, FIG. 1G, FIG. 1H, FIG. 11, FIG. 1J, FIG. 2B, FIG. 3A, FIG. 3B, FIG. 3C, FIG. 3D, FIG. 3F, FIG. 3G, FIG. 3H, FIG. 4A, FIG. 4B, FIG. 5A, FIG. 5B, FIG. 5H, and/or FIG. 5J herein) to deliver a single dose of medicament to a patient (not shown). According to some embodiments, the BFS connector 650 may comprise and/or define an interior volume, mounting socket, or BFS port 650-1 extending axially inward from a first end of the BFS connector 650. In some embodiments, the BFS port 650-1 may be shaped to accept and/or retain a neck (and/or other portion) of a BFS vial, e.g., by defining various internal dimensions and/or features. The BFS port 650-1 may comprise and/or define, for example, a valve seat 650-2 in communication with a canula bore 650-3 extending from the valve seat 650-2 to a second or distal end of the BFS connector 650. According to some embodiments, the BFS port 650-1 (and/or the valve seat 650-2) may comprise and/or define various features that are specially located, sized, and/or shaped to accept and/or engage with a mounting flange (not shown in FIG. 6A, FIG. 62, FIG. 6C, FIG. 6D, FIG. 6E, FIG. 6F, FIG. 6G, and FIG. 6H; e.g., the mounting flanges 116, 216, 316, 416, 516e of FIG. 1A, FIG. 1C, FIG. 1E, FIG. 1F, FIG. 1G, FIG. 1H, FIG. 11, FIG. 1J, FIG. 2B, FIG. 2C, FIG. 2F, FIG. 2H, FIG. 21, FIG. 3A, FIG. 3B, FIG. 3C, FIG. 3D, FIG. 3F, FIG. 3H, FIG. 4A, FIG. 4B, FIG. 5B, FIG. 5C, FIG. 5D, and/or FIG. 5G herein). In some embodiments, the BFS connector 650 may comprise an exterior flange 652 and/or the BFS port 650-1 may comprise and/or define internal threads 656. In some embodiments, the threads 656 may be sized and/or shaped to accept and/or guide the mounting flanges of a cooperatively configured BFS vial such that the BFS vial may be rotationally engaged and "threaded" into the BFS port 650-1 (e.g., despite lacking external threads itself). In some embodiments, the BFS port 650-1 and/or the valve seat 650-2 thereof may comprise, define, and/or be coupled to one or more protrusions, features, and/or shear projections 660 that extend radially inward from an outer diameter of the BFS port 650-1 and/or the valve seat 650-2. As depicted in FIG. 6D and FIG. 6H, for example, a pair of symmetrical opposing shear projections 660 may be provided within the valve seat 650-2, such that portions of the valve seat 650-2 end-surface and/or volume are unimpeded while those areas/volumes corresponding to the shear projections 660 are radially impeded. In some embodiments, the shear projection(s) 660 may comprise and/or encompass between one eighth (1/8) and one third (1/3) of the area and/or volume of the valve seat 650-2. According to some embodiments, the shear projection 660 may comprise and/or define a shear surface 660-1 that is configured to resist rotational movement of, e.g., an inserted and rotated portion of a BFS vial, such as a tear-activation valve (not shown in FIG. 6A, FIG. 6B, FIG. 6C, FIG. 6D, FIG. 6E, FIG. 6F, FIG. 6G, and FIG. 6H; e.g., the tear-activation valve 130, 230, 230ii, 330, 430, 530a-e of FIG. 1A, FIG. 1C, FIG. 1E, FIG. 1G, FIG. 1H, FIG. 11, FIG. 1J, FIG. 2B, FIG. 2D, FIG. 2F, FIG. 2J, FIG. 2K, FIG. 2L, FIG. 2M, FIG. 2N, FIG. 20, FIG. 2P, FIG. 2Q, FIG. 3A, FIG. 3B, FIG. 3C, FIG. 3D, FIG. 3F, FIG. 3G, FIG. 3H, FIG. 4A, FIG. 4B, FIG. 5A, FIG. 5B, FIG. 5C, FIG. 5D, FIG. 5F, FIG. 5G, FIG. 5H, FIG. 51, and/or FIG. 5J herein). In some embodiments, the shear surface 660-1 may be oriented normal to a rotational axis of the BFS connector 650. In some embodiments, the shear projection(s) 660 may comprise a piercing, severing, sharpened, pointed, and/or parallel-oriented shear surface 660-1 and/or other feature formed and/or coupled within the valve seat 650-2. According to some embodiments, and as depicted in FIG. 6F, FIG. 6G, and FIG. 6H, an administration member 680 may be coupled to and/or retained by the BFS connector 650. The administration member 680 may be disposed, for example, at least partially within the canula bore 650-3.

In some embodiments, fewer or more components 650-1, 650-2, 650-3, 652, 656, 660, 660-1, 680 and/or various configurations of the depicted components 650-1, 650-2, 650-3, 652, 656, 660, 660-1, 680 may be included in the BFS connector 650 without deviating from the scope of embodiments described herein. In some embodiments, the components 650-1, 650-2, 650-3, 652, 656, 660, 660-1, 680 may be similar in configuration and/or functionality to similarly named and/or numbered components as described herein.

Referring now to FIG. 7A, FIG. 7B, and FIG. 7C, various views of a BFS connector 750 according to some embodiments are shown. In some embodiments, the BFS connector 750 may be a component of and/or comprise an administration assembly that is configured to mate with a BFS vial (not shown in FIG. 7A, FIG. 7B, and FIG. 7C; e.g., the neck 112, 212, 312, 412, 512a-e of FIG. 1A, FIG. 1B, FIG. 1C, FIG. 1D, FIG. 1E, FIG. 1F, FIG. 1G, FIG. 1H, FIG. 11, FIG. 1J, FIG. 2B, FIG. 3A, FIG. 3B, FIG. 3C, FIG. 3D, FIG. 3F, FIG. 3G, FIG. 3H, FIG. 4A, FIG. 4B, FIG. 5A, FIG. 5B, FIG. 5H, and/or FIG. 5J herein) to deliver a single dose of medicament or other fluid agent to a patient (not shown). According to some embodiments, the BFS connector 750 may comprise and/or define an interior volume, mounting socket, or BFS port 750-1 extending axially inward from a first end of the BFS connector 750. In some embodiments, the BFS port 750-1 may be shaped to accept and/or retain a neck (and/or other portion) of a BFS vial, e.g., by defining various internal dimensions and/or features. The BFS port 750-1 may comprise and/or define, for example, a valve seat 750-2 in communication with a fluid bore 750-3 extending from the valve seat 750-2 to a second or distal end of the BFS connector 750. According to some embodiments, the BFS port 750-1 (and/or the valve seat 750-2) may comprise and/or define various features that are specially located, sized, and/or shaped to accept and/or engage with a mounting flange (not shown in FIG. 7A, FIG. 7B, and FIG. 7C; e.g., the mounting flanges 116, 216, 316, 416, 516e of FIG. 1A, FIG. 1C, FIG. 1E, FIG. 1F, FIG. 1G, FIG. 1H, FIG. 11, FIG. 1J, FIG. 2B, FIG. 2C, FIG. 2F, FIG. 2H, FIG. 21, FIG. 3A, FIG. 3B, FIG. 3C, FIG. 3D, FIG. 3F, FIG. 3H, FIG. 4A, FIG. 4B, FIG. 5B, FIG. 5C, FIG. 5D, and/or FIG. 5G herein).

In some embodiments, the connector 750 may comprise and/or define an interior volume, seat, or hub socket 750-4 disposed at the second or distal end thereof. According to some embodiments, the connector 750 may comprise and/or define a mounting projection, feature, or cone 750-5 that extends axially within (or at least partially within) the hub socket 750-4. In some embodiments, the fluid bore 750-3 may extend through the mounting cone 750-5. In some embodiments, the connector 750 may comprise and/or define an interior radial flange, ridge, or lip 750-6 within the hub socket 750-4. The lip 750-6 may, for example, reduce an interior diameter of the hub socket 750-4 at the position of the lip 750-6 within the hub socket 750-4. The lip 750-6 may, for example, act to retain an exterior flange (not shown) of a needle hub (also not shown; e.g., the needle hub 270 of FIG. 2B, FIG. 2C, FIG. 2D, FIG. 2E, and/or FIG. 2F herein) inserted into the hub socket 750-4.

According to some embodiments, the connector 750 may comprise and/or define one or more of (i) an exterior flange 752, (ii) a sealing flange 754 formed and/or disposed within the BFS port 750-1 and/or (ii) threads 756 formed and/or disposed within the BFS port 750-1. The exterior flange 752 may be configured to mate with and/or retain a cap (not shown), for example. In some embodiments, the threads 756 may be cooperatively sized and shaped to accept and/or guide the mounting flanges of a cooperatively configured BFS vial such that the BFS vial may be rotationally engaged and "threaded" into the BFS port 750-1 (e.g., despite lacking external threads itself). According to some embodiments, the sealing flange 754 may be sized and/or shaped such that, e.g., upon rotational engagement of a BFS vial within the BFS port 750-1, the BFS vial (or a portion thereof, such as an end- seal) pushes against the sealing flange 754, thereby forming a seal to prevent fluid from escaping the BFS port 750-1.

According to some embodiments, the connector 750 may comprise and/or define a shaped edge 758 at the proximal end, e.g., at the opening of the BFS port 750-1. The shaped edge 758, instead of comprising a uniform circumferential surface disposed at a uniform distance from the distal end of the connector 750, for example, may comprise and/or define a track or cam surface that varies in axial distance from the distal end of the connector 750 (and/or from the exterior flange 752). In some embodiments, the shaped edge 758 may comprise and/or define an anti-rotation surface 758-1 that comprises a surface normal to the circumferential plane of the proximal end of the connector 750. The anti-rotation surface 758-1 may be formed and/or defined, for example at a point where the furthest axial distance from the shaped edge 758 to the distal end of the connector 750 (and/or to the exterior flange 752) transitions to the nearest axial distance from the shaped edge 758 to the distal end of the connector 750 (and/or to the exterior flange 752). This transition may define, for example, a notch 758-2, that may be utilized to selectively engage with and/or retain a side flange of a BFS bottle.

In some embodiments, the BFS port 750-1 and/or the valve seat 750-2 thereof may comprise, define, and/or be coupled to one or more protrusions, features, and/or shear projections 760 that extend radially inward from an outer diameter of the BFS port 750-1 and/or the valve seat 750-2. As depicted in FIG. 7B, for example, a pair of symmetrical opposing shear projections 760 may be provided within the valve seat 750-2, such that portions of the valve seat 750-2 end-surface and/or volume are unimpeded while those areas/volumes corresponding to the shear projections 760 are radially impeded. In some embodiments, the shear projection(s) 760 may comprise and/or encompass between one eighth (1/8) and one third (1/3) of the area and/or volume of the valve seat 750-2. According to some embodiments, the shear projection(s) 760 may comprise and/or define a shear surface 760-1 that is configured to resist rotational movement of, e.g., an inserted and rotated portion of a BFS vial, such as a tear-activation valve (not shown in FIG. 3A, FIG. 3B, FIG. 3C, FIG. 3D, FIG. 3E, FIG. 3F, FIG. 3G, and FIG. 3H; e.g., the tear-activation valve 130, 230,230ii, 330, 430, 530a-e of FIG. 1A, FIG. 1C, FIG. 1E, FIG. 1G, FIG. 1H, FIG. 11, FIG. 1J, FIG. 2B, FIG. 2D, FIG. 2F, FIG. 2J, FIG. 2K, FIG. 2L, FIG. 2M, FIG. 2N, FIG. 20, FIG. 2P, FIG. 2Q, FIG. 3A, FIG. 3B, FIG. 3C, FIG. 3D, FIG. 3F, FIG. 3G, FIG. 3H, FIG. 4A, FIG. 4B, FIG. 5A, FIG. 5B, FIG. 5C, FIG. 5D, FIG. 5F, FIG. 5G, FIG. 5H, FIG. 51, and/or FIG. 5J herein). In some embodiments, the shear surface(s) 760-1 may be oriented normal to a rotational axis of the BFS connector 750. In some embodiments, the shear projection(s) 760 may comprise a piercing, severing, sharpened, pointed, and/or parallel-oriented shear surface 760-1 and/or other feature formed and/or coupled within the valve seat 750-2.

In some embodiments, fewer or more components 750-1, 750-2, 750-3, 750-4, 750-5, 750-6, 752, 754, 756, 758, 758-1, 758-2, 760, 760-1 and/or various configurations of the depicted components 750-1, 750-2, 750-3, 750-4, 750-5, 750-6, 752, 754, 756, 758, 758-1, 758-2, 760, 760-1 may be included in the BFS connector 750 without deviating from the scope of embodiments described herein. In some embodiments, the components 750- 1, 750-2, 750-3, 750-4, 750-5,750-6, 752, 754, 756, 758, 758-1, 758-2, 760, 760-1 may be similar in configuration and/or functionality to similarly named and/or numbered components as described herein. According to some embodiments, the BFS connector 750 may comprise an administration member (not shown; e.g., a canula or needle) coupled to, retained by, and/or disposed, at least partially, within the fluid bore 750-3.

and/or FIG. 5J herein) to deliver a single dose of medicament to a patient (not shown). According to some embodiments, the BFS connector 850 may comprise and/or define an interior volume, mounting socket, or BFS port 850-1 extending axially inward from a first end of the BFS connector 850 (e.g., within a body, not separately labeled, thereof). In some embodiments, the BFS port 850-1 may be shaped to accept and/or retain a neck (and/or other portion) of a BFS vial, e.g., by defining various internal dimensions and/or features. The BFS port 850-1 may comprise and/or define, for example, a valve seat 850-2 in communication with a fluid bore 850-3 extending from the valve seat 2850- to a second or distal end of the BFS connector 850. According to some embodiments, the BFS port 850-1 (and/or the valve seat 850-2) may comprise and/or define various features that are specially located, sized, and/or shaped to accept and/or engage with a mounting flange (not shown in FIG. 8A, FIG. 8B, FIG. 8C, FIG. 8D, and FIG. 8E; e.g., the mounting flanges 116, 216, 316, 416, 516e of FIG. 1A, FIG. 1C, FIG. 1E, FIG. 1F, FIG. 1G, FIG. 1H, FIG. 11, FIG. 1J, FIG. 2B, FIG. 2C, FIG. 2F, FIG. 2H, FIG. 21, FIG. 3A, FIG. 3B, FIG. 3C, FIG.

In some embodiments, the BFS port 850-1 and/or the valve seat 850-2 thereof may comprise, define, and/or be coupled to one or more protrusions, features, and/or shear projections 860 that extend radially inward

Turning to FIG. 8A, FIG. 8B, FIG. 8C, FIG. 8D, and FIG. 8E, various views of a BFS connector 850.according to some embodiments are shown. In some embodiments, the BFS connector 850 may be a component of and/or comprise an administration assembly that is configured to mate with a BFS vial or a portion thereof (not shown in FIG. 8A, FIG. 8B, FIG. 8C, FIG. 8D, and FIG. 8E; e.g., the neck 112, 212, 312, 412, 512a-e of FIG. 1A, FIG. 1B, FIG. 1C, FIG. 1D, FIG. 1E, FIG. 1F, FIG. 1G, FIG. 1H, FIG. 11, FIG. 1J, FIG. 3A, FIG. 3B, FIG. 3C, FIG.

3D, FIG. 3F, FIG. 3G, FIG. 3H, FIG. 4A, FIG. 4B, FIG. 5A, FIG. 5B, FIG. 5C, FIG. 5D, FIG. 5G, FIG. 5H, FIG. 51,3D, FIG. 3F, FIG. 3H, FIG. 4A, FIG. 4B, FIG. 5B, FIG. 5C, FIG. 5D, and/or FIG. 5G herein). In some embodiments, the BFS connector 850 may comprise an exterior flange 852 and/or the BFS port 850-1 may comprise and/or define internal threads 856. In some embodiments, the threads 856 may be sized and/or shaped to accept and/or guide the mounting flanges of a cooperatively configured BFS vial such that the BFS vial may be rotationally engaged and "threaded" into the BFS port 850-1 (e.g., despite lacking external threads itself).

According to some embodiments, the BFS connector 850 may comprise and/or define a shaped edge 858 at the proximal end, e.g., at the opening of the BFS port 850-1. The shaped edge 858, instead of comprising a uniform circumferential surface disposed at a uniform distance from the distal end of the BFS connector 850, for example, may comprise and/or define a track or cam surface that varies in axial distance from the distal end of the BFS connector 850 (and/or from the exterior flange 852). In some embodiments, the shaped edge 858 may comprise and/or define an anti-rotation surface 858-1 that comprises a surface normal to the circumferential plane of the proximal end of the connector 850. The anti-rotation surface 858-1 may be formed and/or defined, for example at a point where the furthest axial distance from the shaped edge 858 to the distal end of the BFS connector 850 (and/or to the exterior flange 852) transitions to the nearest axial distance from the shaped edge 858 to the distal end of the BFS connector 850 (and/or to the exterior flange 852). This transition may define, for example, a notch 858-2, that may be utilized to selectively engage with and/or retain a side flange of a BFS bottle.

In some embodiments, the BFS port 850-1 and/or the valve seat 850-2 thereof may comprise, define, and/or be coupled to one or more protrusions, features, and/or shear projections 860 that extend radially inward from an outer diameter of the BFS port 850-1 and/or the valve seat 850-2 (e.g., the shear projections 860 extending normal to a planar surface of the valve seat 850-2). As depicted in FIG. 8D and FIG. 8E, for example, a pair of symmetrical opposing shear projections 860 may be provided within the valve seat 850-2, such that portions of the valve seat 850-2 end-surface and/or volume are unimpeded (the area "A" in FIG. 8D) While those areas/volumes corresponding to the shear projections 860 are radially impeded. In some embodiments, the shear projection(s) 860 may comprise and/or encompass two (2) opposing hemispherical portions of the area and/or volume of the valve seat 850-2, e.g., leaving a substantially rectangular strip or portion of the valve seat 850-2 ("A") unimpeded. The unimpeded portion(s) of the valve seat 850-2 may comprise and/or define a linear and/or rectangular shape extending along a diameter of the valve seat 850-2 and cooperatively shaped to receive a portion of a tear-activation valve (not shown) such as a linear/rectangular-shaped second shaped portion or "tab" thereof. According to some embodiments, the shear projection(s) 860 may comprise and/or define a shear surface 860-1 that is configured to resist rotational movement of, e.g., an inserted and rotated portion of a BFS vial, such as a tear-activation valve and/or portion thereof (not shown in FIG. 8A, FIG. 8B, FIG. 8C, FIG. 8D, and FIG. 8E; e.g., the tear-activation valve 130, 230, 230ii, 330, 430, 530a-e and/or second shaped portion 230-2, 330-2, 430-2, 530-2d, 530-2e of FIG. 1A, FIG. 1C, FIG. 1E, FIG. 1G, FIG. 1H, FIG. 11, FIG. 1J, FIG. 2B, FIG. 2D, FIG. 2F, FIG. 2J, FIG. 2K, FIG. 2L, FIG. 2M, FIG. 2N, FIG. 20, FIG. 2P, FIG. 2Q, FIG. 3A, FIG. 3B, FIG. 3C, FIG. 3D, FIG. 3F, FIG. 3G, FIG. 3H, FIG. 4A, FIG. 4B, FIG. 5A, FIG. 5B, FIG. 5C, FIG. 5D, FIG. 5F, FIG. 5G, FIG. 5H, FIG. 51, and/or FIG. 5J herein). In some embodiments, the shear surface 860-1 may be oriented normal to a rotational axis of the BFS connector 850. In some embodiments, the shear projection(s) 860 may comprise a piercing, severing, sharpened, pointed, and/or parallel-oriented shear surface 860-1 and/or other feature formed and/or coupled within the valve seat 850-2

. In some embodiments, fewer or more components 850-1, 850-2, 850-3, 852, 856, 858, 858-1, 858-2, 860, 860-1 and/or various configurations of the depicted components 850-1, 850-2, 850-3, 852, 856, 858, 858-1, 858-2, 860, 860-1 may be included in the BFS connector 850 without deviating from the scope of embodiments described herein. In some embodiments, the components 850-1, 850-2, 850-3, 852, 856, 858, 858-1, 858-2, 860, 860-1 may be similar in configuration and/or functionality to similarly named and/or numbered components as described herein.

### V. BFS Connector Tear-Activation Valve Methods and Processes

In some embodiments, one or more of the systems 100, 200, 500, BFS vials 110, 210, 210ii, 310, 410, 510a-e, and/or BFS connectors 150, 250, 650, 750, 850 herein may be utilized to perform and/or effectuate one or more methods and/or processes. The process (and/or any diagrams and/or flow diagrams related thereto) described herein do not necessarily imply a fixed order to any described and/or depicted actions, steps, and/or procedures, and embodiments may generally be performed in any order that is practicable unless otherwise and specifically noted. While the order of actions, steps, and/or procedures described and/or depicted herein is generally not fixed, in some embodiments, actions, steps, and/or procedures may be specifically performed in the order listed, depicted, and/or described and/or may be performed in response to any previously listed, depicted, and/or described action, step, and/or procedure.

According to some embodiments, a BFS vial (e.g., including a tear-activation valve component) and/or administration assembly (or a plurality of BFS vials and/or administration assemblies, e.g., a shipment) may be provided for administering a fluid agent to a target such as a human or animal patient. In some embodiments, a partially pre-assembled and/or pre-engaged combination of at least one BFS vial and at least one corresponding administration assembly may be provided subsequent to a BFS manufacturing and pre-assembly process. The BFS manufacturing process may comprise, for example, operation of at least one BFS manufacturing machine by heating and extruding a parison material, applying blown air and/or vacuum pressure to form the parison into at least one mold cavity (e.g., of a primary or body mold), filling the formed vial(s) with a fluid, and sealing the vial(s) (e.g., via a head or sealing mold). According to some embodiments, either the main/body mold or the head/sealing mold may form the tear-activation valve component and/or portion of the BFS vial. In some embodiments, and administration assembly may then be attached to each vial to a first degree, e.g., defining a transport or pre- activation state. Threads of a connector element of the administration assembly may, for example, be partially engaged with a BFS vial, and the BFS vial may accordingly be partially inserted into the connector/administration assembly. According to some embodiments, the first degree or state of coupling/connection may cause the tear- activation valve of the BFS vial to become seated in a valve seat of the connector/administration assembly.

In some embodiments, and presumably immediately prior to usage (e.g., injection), the BFS vial and the administration assembly may be transitioned to an activated or second state or degree of coupling by continuing to engage or insert the BFS vial into the connector. As described herein, for example, continued threading of an exterior mounting flange of the BFS vial into threads of the connector may cause a shear projection within and/or extending into the valve seat to engage with the tear-activation valve (and/or a portion thereof). Force exerted by the shear projection(s) on the tear-activation valve (e.g., rotational shear force) may cause the tear-activation valve to rip or tear from the BFS vial (partially or completely), e.g., exposing the fluid within the BFS vial to the interior of the connector/administration assembly. In some embodiments, the continued engagement/advancement of the coupling may cause a neck portion of the BFS vial to become inserted into a cavity of the tear-activation valve. According to some embodiments, squeeze force (e.g., radially inward force) applied to a compressible reservoir of the BFS vial may then force the fluid from the BFS vial, through the cavity of the tear-activation valve, and through the connector to an administration element (e.g., a needle, dropper, or spray nozzle) of the administration assembly.

According to some embodiments, an attempt to refill the BFS vial after dispensing of the original fluid may cause the tear-activation valve to block the pathway into the BFS vial, thereby preventing refilling. In some embodiments, removal of the BFS vial from the administration assembly may cause the tear-activation valve to fully separate from the BFS vial (e.g., in the case that it was still attached) such that it remains lodged in the valve seat even if the BFS vial is removed from the connector. Accordingly, an attempt to reuse the connector/administration assembly may be prevented because any new BFS vial would collide with the previously- used tear-activation valve and would be unable to seat properly within the connector/administration assembly. In these manners, for example, the BFS vial with the tear-activation valve and cooperatively coupled and configured administration assembly may comprise AD functionality that substantially inhibits or prevents re-use.

### VI. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention(s) nor a listing of features of the invention(s) that must be present in all embodiments.

Neither the Title (set forth at the beginning of the first page of this patent application) nor the Abstract (set forth at the end of this patent application) is to be taken as limiting in any way as the scope of the disclosed invention(s).

The term "product" means any machine, manufacture and/or composition of matter as contemplated by 35 U.S.C. §101, unless expressly specified otherwise.

The terms "an embodiment", "embodiment", "embodiments", "the embodiment", "the embodiments", "one or more embodiments", "some embodiments", "one embodiment" and the like mean "one or more (but not all) disclosed embodiments", unless expressly specified otherwise.

A reference to "another embodiment in describing an embodiment does not imply that the referenced embodiment is mutually exclusive with another embodiment (e.g., embodiment described before the referenced embodiment) unless expressly specified otherwise.

The terms "a", "an" and "the" mean "one or more", unless expressly specified otherwise.

The term "plurality" means "two or more", unless expressly specified otherwise.

The term "herein" means "in the present application, including anything which may be incorporated by reference", unless expressly specified otherwise.

The phrase "at least one of", when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel,(iv) a widget and a car, (v) a widget and a wheel, (vi) a car and a wheel, or (vii) a widget, a car and a wheel.

The phrase "based on" does not mean "based only on", unless expressly specified otherwise. In other words, the phrase "based on" describes both "based only on" and "based at least on.

Where a limitation of a first claim would cover one of a feature as well as more than one of a feature (e.g., a limitation such as "at least one widget" covers one widget as well as more than one widget), and where in a second claim that depends on the first claim, the second claim uses a definite article "the" to refer to the limitation (e.g., "the widget"), this does not imply that the first claim covers only one of the feature, and this does not imply that the second claim covers only one of the feature (e.g., "the widget" can cover both one widget and more than one widget..

Each process (whether called a method, algorithm or otherwise) inherently includes one or more steps, and therefore all references to a "step" or "steps" of a process have an inherent antecedent basis in the mere recitation of the term 'process' or a like term. Accordingly, any reference in a claim to a'step' or 'steps' of a process has sufficient antecedent basis.

When an ordinal number (such as "first", "second", "third" and so on) is used as an adjective before a term, that ordinal number is used (unless expressly specified otherwise) merely to indicate a particular feature, such as to distinguish that particular feature from another feature that is described by the same term or by a similar term. For example, a "first widget" may be so named merely to distinguish it from, e.g., a "second widget". Thus, the mere usage of the ordinal numbers "first" and "second" before the term "widget" does not indicate any other relationship between the two widgets, and likewise does not indicate any other characteristics of either or both widgets. For example, the mere usage of the ordinal numbers "first" and "second" before the term "widget" (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers "first" and "second" before the term "widget" does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality / features. Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the relevant embodiment, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list "a computer, a laptop, a PDA" does not imply that any or all of the three items of that list are mutually exclusive and does not imply that any or all of the three items of that list are comprehensive of any category.

Headings of sections provided in this patent application and the title of this patent application are for convenience only, and are not to be taken as limiting the disclosure in any way. "Determining" something can be performed in a variety of manners and therefore the term "determining" (and like terms) includes calculating, computing, deriving, looking up (e.g., in a table, database or data structure), ascertaining and the like

The terms "including", "comprising" and variations thereof mean "including but not limited to", unless expressly specified otherwise. As used herein, "comprising" means "including," and the singular forms "a" or "an" or "the" include plural references unless the context clearly dictates otherwise. The term "or" refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the relevant embodiment, and does not imply that the illustrated process is preferred.

The present disclosure provides, to one of ordinary skill in the art, an enabling description of several embodiments and/or inventions. Some of these embodiments and/or inventions may not be claimed in the present application but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention(s) as defined by the claims appended hereto.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention(s) as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions. as defined and used herein, should be understood to control ver dictionary definitions in documents incorporated by reference and/or ordinary meanings of the defined terms.

The indefinite articles "a" and "an," as used herein in the specification and in the claims, unless clearly indicated to the contrary, should be understood to mean "at least one." This rule applies even within the body of a claim where a first instance of an element utilizes "a" or "an" and a second or subsequent instance of the element necessarily utilizes (e.g., for purposes of proper grammar and required antecedent basis) the definite article "the" to refer to the element. The use of the definite article "the" does not limit the element to a single object merely because it is utilized to refer back to a previous mention of the element. The original reference to the element controls with respect to the plurality (or lack thereof) of the element.

The phrase "and/or," as used herein in the specification and in the claims, should be understood to mean "either or both" of the elements so conjoined, i.e., elements that are conjunctively present in some cases and disjunctively present in other cases. Other elements may optionally be present other than the elements specifically identified by the "and/or" clause, whether related or unrelated to those elements specifically identified, unless clearly indicated to the contrary.

Reference throughout this specification to "one embodiment" or "an embodiment" means that a particular feature, structure, or characteristic described in connection with the embodiment is included in at least one embodiment. Thus, appearances of the phrases "in one embodiment" or "in an embodiment" in various places throughout this specification are not necessarily all referring to the same embodiment. Furthermore, the particular features, structures, or characteristics may be combined in any suitable manner in one or more embodiments

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term "about." Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word "about" is recited. Whenever "substantially," "approximately," "about," or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as "inner," "outer,", "upper," "lower," "top," "bottom," "interior," "exterior," "left," right," "front," "back," "rear," and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an "upper" part can become a "lower" part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents

Various modifications of the invention(s) and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of the invention(s) in the various embodiments and equivalents thereof.

What is claimed is:

1. A pre-filled, Auto-Disable (AD) medical delivery device, comprising:a Blow-Fill-Seal (BFS) vial comprising:(i) an exterior radial flange;(ii) a collapsible fluid chamber;(iii) a fluid seal at a distal end of the BFS vial; and(iv) a tear-activation valve disposed at the fluid seal, the tear-activation valve comprising an axial projection;an administration assembly comprising:(i) an interior volume comprising an interior seat and at least one flange guide;(ii) an opening at a first end, the opening being in communication with the interior volume;(iii) a fluid channel extending between the interior seat and a second end;(iv) a needle disposed at the second end and in communication with the fluid channel; and(v) a shear projection disposed within the interior seat; andwherein the at least one flange guide is cooperatively configured to accept and guide the exterior radial flange of the BFS vial,wherein the interior seat is cooperatively configured to accept the tear-activation valve, andwherein the shear projection is configured to engage the tear-activation valve at a particular degree of rotation and cause the tear-activation valve to form a hole in the fluid seal, exposing fluid contents of the collapsible fluid chamber to the fluid channel.

2. The pre-filled, AD medical delivery device of claim 1, wherein the at least one flange guide comprises interior threads.

3. The pre-filled, AD medical delivery device of claim 1, wherein the shear projection is configured to engage the tear-activation valve upon a rotational engagement beyond the particular degree of rotation.

4. The pre-filled, AD medical delivery device of claim 1, wherein the tear-activation valve comprises a channel disposed on a distal end thereof.

5. The pre-filled, AD medical delivery device of claim 1, wherein the tear-activation valve comprises a first shaped portion and at least one second shaped portion emanating radially outward therefrom, and wherein the shear projection is cooperatively configured to engage with the second shaped portion.

6. The pre-filled, AD medical delivery device of claim 1, wherein the tear-activation valve comprises a diameter that is less than a diameter of the collapsible fluid chamber.

7. A pre-filled, Auto-Disable (AD) Blow-Fill-Seal (BFS) vial, comprising:a body defining a collapsible fluid chamber pre-filled with a medical fluid;a neck portion extending axially from the body, the neck portion defining an outer radial surface and defining an interior volume in fluid communication with the collapsible fluid chamber;an angled exterior radial flange extending radially outward from the neck portion and being angled with respect to a plane normal to an axis of the BFS vial;a tear-activation valve formed at a distal end of the neck, the tear-activation valve comprising a first shaped portion having a diameter less than a diameter of the neck portion;a fluid seal formed at a junction of the neck portion and the first shaped portion of the tear-activation valve; anda fluid channel feature formed on the outer radial surface of the neck portion and extending axially along a portion of the outer radial surface between the angled exterior radial flange and the fluid seal.

8. The pre-filled, AD BFS vial of claim 7, wherein the tear-activation valve further comprises a second shaped portion extending axially from the first shaped portion.

9. The pre-filled, AD BFS vial of claim 8, wherein the second shaped portion comprises a linear shape extending along a diameter of the tear-activation valve.

10. The pre-filled, AD BFS vial of claim 7, wherein the fluid channel feature comprise a detent.

11. The pre-filled, AD BFS vial of claim 7, wherein the fluid channel feature comprises a plurality of cooperatively spaced ridges.

12. The pre-filled, AD BFS vial of claim 7, wherein the angled exterior radial flange comprises three angled exterior radial flanges spaced axially from each other.

13. The pre-filled, AD BFS vial of claim 7, wherein the neck portion comprises a wall thickness that decreases proximate to the fluid seal.

14. A pre-filled, Auto-Disable (AD) Blow-Fill-Seal (BFS) administration assembly comprising:a cylindrical body comprising a proximate end and a distal end, the cylindrical body defining an interior volume comprising an interior valve seat and at least one flange guide;an opening at the proximate end, the opening being in communication with the interior volume;a fluid channel extending between the interior valve seat and the distal end;a needle disposed at the distal end and in communication with the fluid channel; anda shear projection disposed within the interior seat, the shear projection defining at least one shear surface disposed normal to a surface of the interior valve seat, and the shear projection occupying a first portion of the interior valve seat with a second portion of the interior valve seat remaining unimpeded by the shear projection.

15. The pre-filled, AD BFS administration assembly of claim 14, wherein the second portion of the interior valve seat comprises a rectangular shape extending across a diameter of the interior valve seat.

16. The pre-filled, AD BFS administration assembly of claim 14, further comprising:a shaped edge formed along a circumference of the opening at the proximate end, the shaped edge defining an anti-rotation surface.
